SEQUENCE LISTING (12 sequences)

SEQ ID NO: 1 - protein, 10 aa, Artificial Sequence
  Description of Artificial Sequence Synthetic His tag
  SITE (1)..(10): This sequence may encompass 2-10 residues
HHHHHHHHHH

SEQ ID NO: 2 - DNA, 21 bp, Artificial Sequence
  Description of Artificial Sequence Synthetic oligonucleotide
taactgactaataagggtacc

SEQ ID NO: 3 - DNA, 34 bp, Artificial Sequence
  Description of Artificial Sequence Synthetic oligonucleotide
tacaataaacagggtaccgtgtgtgtgtaagctt

SEQ ID NO: 4 - DNA, 10 bp, Artificial Sequence
  Description of Artificial Sequence Synthetic oligonucleotide
cgccgccacc

SEQ ID NO: 5 - protein, 6 aa, Artificial Sequence
  Description of Artificial Sequence Synthetic 6xHis tag
HHHHHH

SEQ ID NO: 6 - DNA, 24 bp, Artificial Sequence
  Description of Artificial Sequence Synthetic oligonucleotide
gactacaaagacgatgacgacaag

SEQ ID NO: 7 - DNA, 2806 bp, Artificial Sequence
  Description of Artificial Sequence Synthetic polynucleotide
ggtaccctcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagtgtacgagctcgatcactagtccagtgtggatcgatcgccgccaccatgcagggaccaacactggccgtgctgggggctctgctggctgtggctgtctccctgttccccgagtgcaaggaaggatttcagtacgccatcagcaaggaccggaaaattggaccactgggaccagagtccctgaccacaacttggcacctgcccaccaagaaaatcgtggactctatggtgcaggtctggtgcgatggcaagaacctgaaaattctggagacatgtactaaggaggagagatacctggtggctgtccacgagcgcgctctgtctaccagtgccgagttcatgcagatcagctccggaacaaagggccctgaagtgatcgacatgcacgacgatttcgaatttggcctgtgcccctgtgatagtaagcctgtgatgcgcggaaaattcaacgcttcactgctgaatggccctgcctttcagatggtgtgcccacaggggtggaccggaacaatcgagtgtattctggctaaccaggacacactggataccacagtggtccggacttaccggaggactaccccttttcagcgcagaaagtggtgcacctatgagaaaatcattggcgaggacatccacgagtgcatcctgggcgggaattggacctgtatcacaggcgaccattctaagctgaaagatgggccaattaagaaatgcaagtggtgtggctacgacttctttgatagtgagggactgcctcattatccaatcggcaaatgtatgctgtcaaacgaaagcgggtacagatatgtggacgatactagctgcgatcgaggaggagtggctatcgtcccaactgggaccctgaagtgtaggatcggaaaagctaccgtgcaggtcattgccacaaatactgacctgggaccaatgccttgctccccagatgaagtgatcgcttctgagggacctgtcgaaaagactgcctgtaccttcaactactccaagacactgccaaacaagtactatgagccccgagaccggtacttccagcagtatatgctgaagggggaatggcagtactggtttgacctggataccgtggaccaccataaggattacttctcagagtttatcgtgattgccgtggtcgctctgctggggggaaagtacgtgctgtggctgctggtcacctatatgatcctgagtgaacagatggccatgggcatgcatggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttggtacc

SEQ ID NO: 8 - DNA, 4028 bp, Artificial Sequence
  Description of Artificial Sequence Synthetic polynucleotide
gcggccgcaggcaacggggcctgctcccgcgcaggagcacgtggtgctcaagatcggggcctcggcctctacgctggccgaggctatgctactgcgaaccttggaccacgccaacgtggtcaagctgaaggccgtgctcttccacggggagctggtgtgcgtggtgctggcgcgctaccgcgaggacctgcacacgcacctctggagaatcaaccgcccgctggcgctccccgcggcgctggcggtgacgcgggccgtgctgcggggcctcgcgtacctgcactcccgccggatcgctcaccgggacgtcaaaacggaaaacgtcttcctcaacggcccaggcgacgtgtgcctgggcgactttggcgcggcacacgggccggtcaccgagccccgctactacggcctggccggcaccctggagacgaactcgccagagctgctggcgcgcgcgcgctacgactgccgcacggacgtgtggagcgcgggcgtcgtcgcgtacgagatgctggcatacccgcgcgcgctgttcgacagccccgcgggcccgcagggcgaggacgccgaggcatcgggcccgccgacgatcttgggcgaccgcgactgcgcccggcagctgctccgcgtgattcgccggctggccgtgcacgccgaagagtttccacccagccccactgaccggctgacccgcaacttcaagcgccacgcgagcacgcgccgagagccgcacagcccgtaccgctgcctggcggtgctccggctgccctgcgacgccgaccgcctcctacaccagatgctgacctttgactttcgcgcgcgccccaccgccgcggagctgctggagcaccccgtcttcggtgcggcctcggggtagccccgggggtttcccgcaaaactgaggcatataaggcgcgggcaccggcaagtttggcatccacacttcgcgctgtggacacgagagcgaacgcgagcgaacgcgagcgcaagcgcgagcacacgactgcgatctacaataaacagggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtgccgccaccatgcagggacctactctggctgtgctgggggctctgctggctgtggctgtctctctggaaaacattactcagtggaatctgatggacaatggcaccgaagggatccagcaggccatgtttctgcgaggcgtgaaccggtctctgcacgggatctggcctgagaagatttgcaccggcgtgccaacacatctggccactgactacgagctgaaggaaattgtcgggatgatggatgctagtgagaaaactaattatacctgctgtcgcctgcagagacacgaatggaacaagcatgggtggtgtaattggttccacatcgagccatggatttggctgatgaacaaaacacagaacaatctgactgagggacagccactgagggaatgcgctgtgacctgtcgctacgacaaggagaccgaactgaatatcgtcacacaggctcgggataggcctaccacactgaccggctgcaagaaagggaaaaacttcagctttgccggcgtgatcctggacggaccatgcaacttcaaggtgagcgtcgaggacgtgctgttcaaggaacacgattgtggaaacatgctgcaggaaacagccatccagctgctggatggcgctacaaatactattgagggagctcgagtgggaaccgctaagctgactacctggctgggcaaacagctgggaatcctgggcaagaaactggagaacaagtctaaagcctggtttggggcccatgctatggcccccactagaccccctaacaccgctacacgaccttggcagcacgtggacgccattaaggaggctctgagcctgctgaaccatagctccgacactgatgccgtgatgaatgacaccgaggtggtctccgaaaagtttgattctcaggagcccacctgtctgcagacacggctgaagctgtacaaaaacgggctgcagggatcactgaccagcctgatgggatccctgactatgatggctacccactatgagaagcattgcccacccacacctgaaactagttgtggcacccagttcatcagcttcaagaacttcaaagaagacctgaaagagttcctgtttattattccatttgactgttgggagcctgcccagaaggactacaaagacgatgacgacaagtaactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagcttggacgcttcgcgcggctgcgccctggccctcttggacatggcggagacggtggtgcccggcggaccgcgagccgccgacgtcgtcgacgtcggctgggcttaccaagacggggactgcatggtgcctctggcatatcgccagtactttaactgcacggggggcgcgctgcccggccaaaacgtctgcgccgggctctctgagacccgcatccgcggtggctttggaacctccgactacgcgctctacgggacgtcgctagtactgcgccccggcctgtacgaccgcgggacctacatctacttccttggatacggcccagacgacatctacgtgggcagcgtcacgctcatggtgggcgccgacatccacaaatacccctgcgggctggaccgagggctcggtgtggccctgcaccacaagagcggaccggcccgacctctgacagaggacgacgccaccggcgactgggcctgcggctgcttccccgcccttgttgaggttgacgcggtgtggggcaacgtaagcgccgcagagctgggcctggccgacccgatcgactacgccgacgaagggggtgaggtcgaagtgctcgaggacgaagccgggagcgccagcggaaacctgccgcaggacgaccccgaccccgacctcgcagattgccggaccgtcgggctctttagcgaaagcgacatgttccggaccgccagcgggcccgaatcgctgctgatcggcgccgttgccaaggacgtcctgacggtgcccctcaatctgccgcccggccgctcttacgaggccctgcgaaacgcatcgctggagtgcaactcccgcccgcgcgagaccggcgacgcagcggtggtggtgatgtctctccaggagcccgctcgcctcgagcgccgccccgatgcccgcgccaccgatccggagtttgggctctttggcctgcccgatgaccccgccgtgcggcgcggcattctcatcggcctcgcgatcgctctgctgatgcat

SEQ ID NO: 9 - protein, 22 aa, Artificial Sequence
  Description of Artificial Sequence Synthetic peptide
GSGATNFSLLKQAGDVEENPGP

SEQ ID NO: 10 - DNA, 4459 bp, Artificial Sequence
  Description of Artificial Sequence Synthetic polynucleotide
ggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtgccgccaccatgcagggccccaccctggctgtgctgggcgctctgctggccgtggctgtgagcctgatggaggacccccacctgaggaaccggcccggaaagggccacaactacatcgacggcatgacccaggaggacgccacctgcaagcccgtgacctacgccggcgcctgcagctctttcgacgtgctgctggagaagggcaagttccccctgttccagagctacgcccaccacaggaccctgctggaggccgtgcacgacaccatcatcgccaaggccgacccccccagctgcgacctgctgtctgcccacggcaacccctgcatgaaggagaagctggtcatgaagacccactgccccaacgactaccagtccgcccactacctgaacaacgacggcaagatggccagcgtgaagtgcccccccaagtacgagctgaccgaggactgcaacttctgccggcagatgaccggcgcctccctgaagaagggctcctaccccctgcaggacctgttctgccagagctccgaggacgacggcagcaagctgaagaccaagatgaagggcgtgtgcgaggtgggcgtgcaggccctgaagaagtgcgacggccagctgtccaccgcccacgaggtggtgcccttcgccgtgttcaagaactccaagaaggtgtacctggacaagctggacctgaagaccgaggagaacctgctgcccgactccttcgtgtgcttcgagcacaagggccagtacaagggcaccatggacagcggccagaccaagagggagctgaagagcttcgacatctcccagtgccccaagatcggcggccacggctccaagaagtgcaccggcgacgccgccttctgcagcgcttacgagtgcaccgcccagtacgccaacgcctactgcagccacgccaacggctccggcatcgtgcagatccaggtgagcggcgtgtggaagaagcccctgtgcgtgggctacgagcgggtggtggtgaagcgggagctgtccgccaagcccatccagcgggtggagccctgcaccacctgcatcaccaagtgcgagccccacggcctggtggtgcggtctaccggattcaagatctccagcgccgtggcctgcgccagcggagtgtgtgtgaccggctcccagtcccccagcaccgagatcaccctgaagtaccccggcatcagccagagcagcggcggcgatatcggcgtgcacatggcccacgacgaccagtccgtgagctccaagatcgtggcccactgccccccccaggacccttgtctggtgcacgactgcatcgtgtgcgcccacggcctgatcaactaccagtgccacaccagggtgctgaagtgcctgaagatcgccccccggaaggtgctgaaccccctgatgtggatcaccgccttcatccggtggatctacaagaagatggtggcccgggtggccgacaacatcaaccaggtgaaccgggagatcggctggatggagggcggccagctggtgctgggcaaccctgctcccatcccccggcacgctcccatccctagaatggcccccaccaggccccccaacaccgctacaaggccctggcagcacgtggacgccatcaaggaggccctgagcctgctgaaccactcctccgacaccgacgccgtgatgaacgacaccgaggtggtgagcgagaagttcgactcccaggagcccacctgcctgcagacccggctgaagctgtacaagaacggcctgcagggctccctgacctccctgatgggctccctgacaatgatggccacccactacgagaagcactgcccccctacccccgagacctcctgcggaacccagttcatctccttcaagaacttcaaggaggacctgaaggagttcctgttcatcatccccttcgactgctgggagcccgcccagaaggactacaaggacgacgacgacaagggcagcggcgccaccaacttcagcctgctgaagcaggccggcgacgtggaggagaaccccggacctatgtgcagcgagctgatccaggcctcctcccggatcaccacctgcagcaccgagggcgtgaacaccaagtgcaggctgtccggcaccgccctgatccgggctggaagcgtgggcgctgaggcctgtctgatgctgaagggcgtgaaggaggaccagaccaagttcctgaagatcaagaccgtgagctccgagctgagctgcagggagggccagagctactggaccggctccttctcccccaagtgcctgtccagccggcggtgccacctggtgggagagtgccacgtgaacaggtgcctgagctggcgggacaacgagaccagcgccgagttcagcttcgtgggcgagtccaccaccatgcgggagaacaagtgcttcgagcagtgcggcggctggggctgcggatgtttcaacgtgaaccccagctgcctgttcgtgcacacctacctgcagtccgtgcggaaggaggccctccgggtgttcaactgcatcgactgggtgcacaagctgaccctggagatcaccgacttcgacggctccgtgtccaccatcgacctgggcgcctcctcctcccggttcaccaactggggcagcgtgtccctgtccctggacgccgagggcatctccggcagcaacagcttcagcttcatcgagagccccggcaagggctacgccatcgtggacgagcccttcagcgagatccccaggcagggcttcctgggcgagatcaggtgcaactccgagagctccgtgctgagcgcccacgagagctgcctgagggcccctaacctgatcagctacaagcccatgatcgaccagctggagtgcaccaccaacctgatcgaccccttcgtggtgttcgagcggggcagcctgccccagaccagaaacgacaagaccttcgccgccagcaagggcaaccggggcgtgcaggctttcagcaagggcagcgtgcaggccgacctgaccctgatgttcgacaacttcgaggtggacttcgtgggcgccgccgtgtcctgcgacgctgctttcctgaacctgaccggctgctactcctgcaacgccggcgccagagtgtgcctgtccatcacctccaccggcaccggctccctgagcgctcacaacaaggacggcagcctgcacatcgtgctgccctccgagaacggcaccaaggaccagtgccagatcctgcacttcaccgtgcccgaggtggaggaggagttcatgtacagctgcgacggcgacgagcggcccctgctggtgaagggaaccctgatcgccatcgacccctttgacgaccggcgggaggccggaggagagtctaccgtggtgaaccccaagtccggcagctggaacttcttcgactggttctccggcctgatgtcctggttcggcggccccctgaagaccatcctgctgatctgcctgtacgtggccctgagcatcggcctgttcttcctgctgatctacctgggccggaccggcctgtccaagatgtggctggccgccaccaagaaggccagcggtaagcctatccctaaccctctcctcggtctcgattctacgcgtaccggttaactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaagctt

SEQ ID NO: 11 - protein, 588 aa, Artificial Sequence
  Description of Artificial Sequence Synthetic polypeptide
MQGPTLAVLGALLAVAVSLMEDPHLRNRPGKGHNYIDGMTQEDATCKPVTYAGACSSFDVLLEKGKFPLFQSYAHHRTLLEAVHDTIIAKADPPSCDLLSAHGNPCMKEKLVMKTHCPNDYQSAHYLNNDGKMASVKCPPKYELTEDCNFCRQMTGASLKKGSYPLQDLFCQSSEDDGSKLKTKMKGVCEVGVQALKKCDGQLSTAHEVVPFAVFKNSKKVYLDKLDLKTEENLLPDSFVCFEHKGQYKGTMDSGQTKRELKSFDISQCPKIGGHGSKKCTGDAAFCSAYECTAQYANAYCSHANGSGIVQIQVSGVWKKPLCVGYERVVVKRELSAKPIQRVEPCTTCITKCEPHGLVVRSTGFKISSAVACASGVCVTGSQSPSTEITLKYPGISQSSGGDIGVHMAHDDQSVSSKIVAHCPPQDPCLVHDCIVCAHGLINYQCHTRVLKCLKIAPRKVLNPLMWITAFIRWIYKKMVARVADNINQVNREIGWMEGGQLVLGNPAPIPRHAPIPRMAPTRPPNTATRPWQHVDAIKEALSLLNHSSDTDAVMNDTEVVSEKFDSQEPTCLQTRLKLYKNGLQGSL

SEQ ID NO: 12 - protein, 530 aa, Artificial Sequence
  Description of Artificial Sequence Synthetic polypeptide
GSGATNFSLLKQAGDVEENPGPMCSELIQASSRITTCSTEGVNTKCRLSGTALIRAGSVGAEACLMLKGVKEDQTKFLKIKTVSSELSCREGQSYWTGSFSPKCLSSRRCHLVGECHVNRCLSWRDNETSAEFSFVGESTTMRENKCFEQCGGWGCGCFNVNPSCLFVHTYLQSVRKEALRVFNCIDWVHKLTLEITDFDGSVSTIDLGASSSRFTNWGSVSLSLDAEGISGSNSFSFIESPGKGYAIVDEPFSEIPRQGFLGEIRCNSESSVLSAHESCLRAPNLISYKPMIDQLECTTNLIDPFVVFERGSLPQTRNDKTFAASKGNRGVQAFSKGSVQADLTLMFDNFEVDFVGAAVSCDAAFLNLTGCYSCNAGARVCLSITSTGTGSLSAHNKDGSLHIVLPSENGTKDQCQILHFTVPEVEEEFMYSCDGDERPLLVKGTLIAIDPFDDRREAGGESTVVNPKSGSWNFFDWFSGLMSWFGGPLKTILLICLYVALSIGLFFLLIYLGRTGLSKMWLAATKKAS

(19) United States
(12) Patent Application Publication
(10) Pub. No.: US 2024/0084325 A1
(43) Pub. Date: Mar. 14, 2024

(54) BOVINE HERPESVIRUS TYPE 1 (BoHV-1) QUADRUPLE GENE DELETED MUTANT

(71) Applicant: Shafiqul I. Chowdhury, Baton Rouge, LA (US)

(72) Inventor: Shafiqul I. Chowdhury, Baton Rouge, LA (US)

(73) Assignee: The Boards of Supervisors of Louisiana State University and Agricultural and Mechanical College, Baton Rouge, LA (US)

(21) Appl. No.: 18/268,553

(22) PCT Filed: Dec. 21, 2021
(86) PCT No.: PCT/US2021/064660

§ 371 Date: Jun. 20, 2023

Related U.S. Application Data
(60) Provisional application No. 63/128,581, filed on Dec. 21, 2020.

(51) Int. Cl. C12N 15/86; A61K 39/12; A61P 31/14; C07K 14/005

(52) U.S. Cl. CPC C12N 15/86; A61K 39/12; A61P 31/14; C07K 14/005; A61K 2039/552; C12N 2710/16734; C12N 2710/16743; C12N 2710/16762

(57) ABSTRACT

The invention relates to a Quadruple Gene Deleted Mutant Bovine Herpesvirus Type 1 (BHV-1 QMV) engineered to express protective antigens derived from viruses associated with infection in livestock. The recombinant vector includes a deletion of a cytoplasmic tail of envelope glycoprotein gE (gE-CT), a truncation of glycoprotein gG, a deletion of envelope protein UL49.5 amino acid residues 30-32, and a deletion of UL49.5 cytoplasmic tail amino acid residues 80-96. The truncation of glycoprotein gG comprises a deletion of amino-terminal amino acid residues 1-67. The recombinant vector can include at least two heterologous antigens inserted therein. Included are methods for creating recombinant vectors, mutant viruses, and vaccines for preventing or reducing symptoms associated with viral infection in livestock, in particular bovine respiratory viral infection

This application claims priority to U.S. Provisional Application No. 63/128,581, filed Dec. 21, 2020, the contents of which are incorporated herein by reference in their entirety.

This invention was made with government support under 2015-67015-2327 and 2020-67016-31543 awarded by the U.S. Department of Agriculture National Institute of Food and Agriculture. The government has certain rights in the invention.

### SEQUENCE LISTING

The instant application contains a Sequence Listing which has been submitted electronically in ASCII format and is hereby incorporated by reference in its entirety. Said ASCII copy, created on Dec. 14, 2021, is named 144240_554137_SL.txt and is 27,286 bytes in size.

### BACKGROUND

### 1. Field of the Invention

The field of the currently claimed embodiments of this invention relate to methods for creating recombinant vectors, mutant viruses, and vaccines for preventing or reducing symptoms associated with bovine respiratory disease complex. In particular, the invention relates to a Quadruple Gene Deleted Mutant Bovine Herpesvirus Type 1 (BoHV-1 QMV).

### 2. Discussion of Related Art

The bovine respiratory disease complex (BRDC) remains a major economic problem for both beef and dairy cattle industries in North America and throughout the world due to calf mortality, treatment expenses, and additional labor incurred. The United States Department of Agriculture National Animal Health Monitoring Service [1] reported. that BRDC affects 12.4% of calves during the pre-weaning period, resulting in 22.5% calf mortality. Additionally, 5.9% of post-weaning animals are eventually diagnosed with BRDC, causing 46.5% of the mortality during that period [1]. The detrimental economic impact of BRDC on the American beef industry is even larger than in the dairy industry. It is the most expensive disease affecting feedlot cattle, and it is estimated to cause losses of approximately (Approx.) one billion dollars per year in the USA [2]. BRDC frequently involves an initial viral respiratory infection followed by a secondary bacterial infection, i.e., Mannheimia haemolytica (M. haemolytica). The initial viral respiratory infection creates a favorable condition for colonization of the lungs, usually by M. haemolytica, resulting in severe pneumonia and death of infected cattle, especially in the feedlots [3].

Among the respiratory viral agents implicated in BRDC, Bovine Herpesvirus Type 1 (BoHV-1 or BHV-1), and Bovine Viral Diarrhea Virus (BVDV) play significant roles because both viruses cause immunosuppression. BoHV-1 downregulates histocompatibility complex class I (MHC-I) [4, 5], causes abortive infection and loss of CD4⁺ T lymphocytes [6], and interferes with the migration of lymphocytes and macrophages to the site of infection by counteracting chemokine activity [7]. BVDV causes leukopenia by infecting and killing lymphocytes and plasma cells [8]. Consequently, initial BoHV-1 and BVDV infections facilitate secondary bacterial infections that lead to death [8-10]. Furthermore, BoHV-1 causes lifelong latency in trigeminal ganglia (TG) with intermittent reactivation and nasal virus shedding [9, 11], whereas BVDV causes persistently infected animals that shed large amounts of virus [12]. As a result, both viruses are maintained in the cattle population [13].

BoHV-1 encodes at least two immunosuppressive envelope proteins, U_{L}49.5 and glycoprotein G (gG). The U_{L}49.5, a non-glycosylated alphaherpesvirus gN homolog, transiently down-regulates MHC-I antigen presentation, which allows the virus to escape T cell recognition and clearance of the infected cells [4, 5, 14]. Similarly, BoHV-1 gG and its homologs in alphaherpesviruses bind to different chemokines secreted by the infected cells and interfere with activated migration of lymphocytes and neutrophils to the site of infection [7]. Consequently, gG disrupts chemokine gradients allowing survival of the infected cell. BVDV is also well-skilled in evading the host's innate and adaptive immunity. Most viruses have only one possibility when they infect a host: either “hit and run” or “infect and persist”. The BVDV has mastered both strategies: i) it counteracts innate immunity, primarily by inhibiting interferon production; ii) it causes a transient leukopenia by infecting and killing the T-lymphocytes and macrophages; iii) it doesn't harm its persistently infected (PI), immunotolerant host for its survival and maintenance; iv) the PI animals shed large amounts of virus, which infects naïve animals, usually subclinically, over a short time; and v) it can mutate rapidly [15, 16]. These properties of both viruses are retained in the current modified-live virus (MLV) vaccine strains [9, 11, 17].

Current vaccinations against these viral diseases utilize polyvalent vaccines containing BoHV-1, BVDV, and BRSV in two formats: MLV or killed virus (KV) [18] vaccines. Additionally, in the EU countries, live and killed BoHV-1 envelope glycoprotein E (gE) gene-deleted vaccines are mandated instead of the traditional MLV and KV vaccines [9, 11]. Like the BoHV-1 wild-type (wt) virus, BoHV-1 MLV vaccines establish lifelong latency in the TG and cause nasal virus shedding following latent reactivation. Similarly, BVDV MLV vaccines can also persistently infect calves and have an added risk of mutating or reverting to virulence. Also, BoHV-1 and BVDV MLV vaccine strains retain the immunosuppressive traits of their respective parental wt strains [9, 11, 17]. Assessment of the effect of widespread BVDV vaccination over several decades is disappointing since this effort has failed to lower BVDV prevalence [15, 19]. This failure is due to the unique biology of BVDV infection, which was

not fully understood for a long time, and is still widely underestimated [20]. Together, these problems associated with the current vaccines have further complicated the BRDC epidemiology in the field and perhaps contributed to outbreaks of abortion and/or respiratory infections in the vaccinated animals [9, 21-25].

While MLV vaccines' safety is of concern, the efficacy of inactivated vaccines is not adequate because they do not induce a cellular immune response. In one instance, an effort to influence the cellular immune response of the BVDV inactivated vaccine resulted in bovine neonatal pancytopenia [26, 27]. BoHV-1 gEΔ marker vaccine is safer than the MLV because it is not transmitted from the vaccinated to the non-vaccinated animals, rarely shed following latency reactivation, and the vaccinated animals are distinguishable from the infected animals. However, based on the protective efficacy measured by neutralization antibody titers following vaccination, the gEA marker vaccine was less efficacious than the traditional MLV, gC-, and TK-deleted vaccines [28].

The Rift Valley Fever Virus (RVFV) is another emerging virus that maintains high biodefense priority based on its threat to livestock, its ability to cause human hemorrhagic fever, and its potential for aerosol spread. RVFV is an RNA virus and a member of the family Bunyaviridae, genus Phlebovirus. RVFV is a mosquito-transmitted viral pathogen of critical livestock species such as sheep, goats, buffalo, and cattle, cause significant economic losses through death, abortion, and decreased milk production. In calves and young lambs, the mortality can be 70% and 100%, respectively, whereas, in older sheep and cattle, the mortality can be up to 30% and 10%, respectively. In pregnant ewes and cows, abortion rates are 100%. In the 1970s, severe outbreaks of RVFV occurred in South Africa (1975) and Egypt (1977, 1978). RVFV has been found widely distributed in sub-Saharan Africa, with epizootic activity affecting animals in Kenya, Tanzania, Zambia, and Uganda. Now, rapid intercontinental commerce and a lack of effective control measures threaten to expand the geographic range of RVFV.

Virally vectored RVFV subunit vaccines such as the capripox virus (CaPV) and the New Castle disease virus (NDV) have been tested in sheep. Both CaPV and NDV vectored vaccines generate neutralizing antibody. However, two vaccine doses are required to induce protection against a virulent RVFV challenge in lambs, and viremia was detected following challenge of the vaccinated animals. One obstacle to the clinical use of RNA-based vaccines such as NDV is their genetic instability that can adversely affect vaccine quality. The production of RVFV-expressing NDV vaccine has to be critically monitored for genetic stability not only under field conditions but also at the vaccine production facility. These validations require complicated testing methods and may not be cost effective. Poxviruses-based vaccines such as CaPV also have their own set of issues. Since they code for 8-9 well-characterized immune-evasion proteins, they exhibit immunosuppressive. While the TK-gene deleted capripoxvirus vector is attenuated, the immune evasion genes remain intact. The effects of these immune evasion genes on the protective efficacy of the vaccine need to be fully characterized, and deletion of one or more of the immunosuppressive genes may be necessary to increase vaccine efficacy before its use in the field. Recently, baculovirus vectored subunit protein vaccine was found to be effective in sheep against a virulent RVFV challenge. However, the vaccine preparation involved protein purification, adjuvant incorporation and required two injections, which could be costly. Importantly, while such vaccines usually induce B cell and CD4+ T cell responses, their CD8+ T cell response, which is crucial for long-lasting immunity, is limited.

There is an unmet need for a vaccine capable of protecting cattle against viral diseases that remains efficacious without having the side effects associated with commercially available vaccines.

### INCORPORATION BY REFERENCE

U.S. Pat. Nos. 8,877,211 and 10,690,669, and U.S. patent application Ser. No. 15/780,900 (Published as U.S. Patent Application Publication No. 2018/0353596 A1) are incorporated herein by reference in their entirety. All other publications and patent applications identified herein are incorporated by reference in their entirety and to the same extent as if each individual publication or patent application was specifically and individually indicated to be incorporated by reference.

### SUMMARY

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The described embodiments of the invention may be modified or varied, without departing from the invention, as appreciated by those skilled in the art in light of the above teachings. Moreover, features described in connection with one embodiment of the invention may be used in conjunction with other embodiments, even if not explicitly stated above. It is therefore to be understood that, within the scope of the claims and their equivalents, the invention may be practiced otherwise than as specifically described.

An embodiment of the invention relates to a bovine herpesvirus-1 (BoHV-1) recombinant vector including a deletion of a cytoplasmic tail of envelope glycoprotein gE (gE-CT), a truncation of glycoprotein gG, a deletion of envelope protein UL49.5 amino acid residues 30-32, and a deletion of UL49.5 cytoplasmic tail amino acid residues 80-96.

An embodiment of the invention relates to the vector above, where the truncation of gG disrupts the chemokine binding ability of glycoprotein gD.

An embodiment of the invention relates to the vector above, where the truncation of glycoprotein gG comprises a deletion of amino-terminal amino acid residues 1-67.

An embodiment of the invention relates to the vector, where the Us3 Poly A sequence is repositioned upstream of the deletion.

An embodiment of the invention relates to the vector above, where the truncated sequence of the glycoprotein Gg is replaced by a sequence having at least 90%, at least 95% or 100% sequence identity with the sequence SEQ ID NO:3.

An embodiment of the invention relates to the vector above, further comprising at least two heterologous antigens inserted therein.

An embodiment of the invention relates to the vector above, where the nucleotide sequence of the heterologous antigens is inserted by homologous recombination therein.

An embodiment of the invention relates to the vector above, where the at least two heterologous antigens are selected from Bovine Viral Diarrhea Virus type 1 (BVDV-1), Bovine Viral Diarrhea Virus type 2 (BVDV-2), Bovine Herpesvirus-1 (BoHV-1), Bovine Respiratory Syncytial Virus (BRSV), and Rift Valley Fever Virus (RVFV).

An embodiment of the invention relates to the vector above, where the at least two heterologous antigens originate from the same virus or different viruses.

An embodiment of the invention relates to the vector above, where the at least two heterologous antigens are viral envelope glycoproteins.

An embodiment of the invention relates to the vector above, where the glycoprotein is mutated as to be expressed as a secreted protein, the glycoprotein being preferably RVFV Gn.

An embodiment of the invention relates to the vector above, where the at least two heterologous antigens are selected from BVDV-2 E2 and BVDV-2 Ems, BRSV F, BRSV G, RVFV Gn, and RFVF Gc.

An embodiment of the invention relates to the vector above, where at least one of the at least two heterologous antigens is expressed as a fusion protein with a fusion partner.

An embodiment of the invention relates to the vector above, where the fusion partner is a cytokine that can potentiate a humoral and/or cellular immunity, preferably the cytokine being GM-CSF.

An embodiment of the invention relates to the vector above, where the fusion partner is selected from a gD signal sequence, a V5 epitope, a histidine tail including 2-10 histidine residues (SEQ ID NO: 1 or SEQ ID NO: 5 or any variations of the His tag can be used as long as it is recognized by commercially available anti-His antibodies), GM-CSF, or any combination thereof.

An embodiment of the invention relates to the vector above, where at least one of the at least two heterologous antigens is expressed from a heterologous promoter.

An embodiment of the invention relates to the vector above, where at least one of the at least two heterologous antigens is expressed from, a heterologous promoter selected from, a viral promoter or a mammalian promoter.

An embodiment of the invention relates to the vector above, where at least one of the at least two heterologous antigens is expressed from a HCMV promotor, an elongation factor 1 alpha promotor, a CMV IE promotor, or a CAG synthetic promotor.

An embodiment of the invention relates to the vector above, comprising a sequence having at least 90%, at least 95% or 100% sequence identity with a sequence selected from SEQ ID NO: 7 in combination with SEQ ID NO: 8 or SEQ ID NO: 10 or SEQ ID NO: 7 in combination with SEQ ID NO: 10.

An embodiment of the invention relates to the vector above, wherein the RVFV antigens comprise a polypeptide having at least 90%, at least 95% or 100% sequence identity with the polypeptide sequences defined as SEQ ID NO: 11 and a polypeptide having at least 90%, at least 95% or 100% sequence identity with the polypeptide sequences defined as SEQ ID NO: 12.

An embodiment of the invention relates to the vector above, where the at least two heterologous antigens is expressed either in the same expression cassette or in different expression cassettes.

An embodiment of the invention relates to the vector above, where the chimeric BVDV-2 E2 antigen is expressed through an expression cassette having a sequence at least 90%, at least 95% or 100% sequence identity with the sequence defined in SEQ ID NO: 7.

An embodiment of the invention relates to the vector above, where the chimeric BVDV-2 Erns-GMSCF sequence having at least 90%, at least 95% or 100% sequence identity with the sequence defined in SEQ ID NO: 8 is inserted therein by homologous recombination.

An embodiment of the invention relates to the vector above, where the chimeric Gn-GMCSF-Gc protein is expressed through an expression cassette having a sequence at least 90%, at least 95% or 100% sequence identity with the sequence defined in SEQ ID NO: 10.

An embodiment of the invention relates to a composition comprising a carrier and at least one of the BoHV-1 recombinant vectors above.

An embodiment of the invention relates to the composition above, formulated for administration by an intranasal route.

An embodiment of the invention relates to a method for treating a mammal having or at risk of having a viral infection, in particular a viral respiratory infection, by administering at least one of the BoHV-1 recombinant vector above to a mammal.

An embodiment of the invention relates to a method for treating a mammal having or at risk of having a viral infection, in particular a viral respiratory infection, by administering at least one BoHV-1 recombinant vector comprising a sequence having at least 90%, at least 95% or 100% sequence identity with a sequence selected from SEQ ID NO: 7 in combination with SEQ ID NO: 8 or SEQ ID NO: 10 or SEQ ID NO: 7 in combination with SEQ ID NO: 10.

An embodiment of the invention relates to the method above, wherein the RVFV antigens comprise a polypeptide having at least 90%, at least 95% or 100% sequence identity with the polypeptide sequence defined as SEQ ID NO: 11 and a polypeptide having at least 90%, at least 95% or 100% sequence identity with the polypeptide sequence defined as SEQ ID NO: 12. An embodiment of the invention relates to the method above where the viral infection is caused by at least one of the viruses selected from BVDV-1, BVDV-2, BoHV-1, BRSV and RVFV.

An embodiment of the invention relates to the method above, where administering at least one BoVH-1 recombinant vector above prevents or reduces the incidence or severity of viral infection in the mammal.

An embodiment of the invention relates to the method above, where administering at least one BoHV-1 recombinant vector above induces humoral and/or cellular immunity.

An embodiment of the invention relates to the method above, where administering at least one BoHV-1 recombinant vector above induces cellular immunity.

An embodiment of the invention relates to the method above, where the mammal is a bovine animal or an experimental animal.

An embodiment of the invention relates to a bovine herpesvirus-1 (BoHV-1) recombinant vector above for use as a vaccine.

An embodiment of the invention relates to a BoHV-1 recombinant vector above for use in the prevention and/or the treatment of viral diseases, preferably viral respiratory diseases, most preferably bovine viral respiratory infection induced by at least one of the viruses selected from BVDV-1, BVDV-2, BoHV-1, BRSV and RVFV.

An embodiment of the invention relates to a live attenuated vaccine for protection against at least one bovine viral disease, in particular a bovine viral respiratory infection, comprising at least one of the BoHV-1 recombinant vector above.

An embodiment of the invention relates to a live attenuated vaccine above, wherein the at least one BoHV-1 recombinant vector comprises a sequence having at least 90%, at least 95% or 100% sequence identity with a sequence selected from SEQ ID NO: 7 in combination with SEQ ID NO: 8 or SEQ ID NO: 10 or SEQ ID NO: 7 in combination with SEQ ID NO: 10.

An embodiment of the invention relates to the live attenuated vaccine above, wherein the RVFV antigens comprise a polypeptide having at least 90%, at least 95% or 100% sequence identity with the polypeptide sequence defined as SEQ ID NO: 11 and a polypeptide having at least 90%, at least 95% or 100% sequence identity with the polypeptide sequence defined as SEQ ID NO: 12.

An embodiment of the invention relates to the live attenuated vaccine above, where the bovine viral respiratory infection is caused by at least one of the viruses selected from BVDV-1, BVDV-2, BoHV-1, BRSV and RVFV.

An embodiment of the invention relates to the vaccine above and a pharmaceutically acceptable vehicle or adjuvant.

An embodiment of the invention relates to a method of vaccinating a cow against a BVDV infection, said method comprising inoculating the cow with the vaccine of above

An embodiment of the invention relates to the method above, where the vaccination results in prevention or reduction of the symptoms associated with a BVDV-1 infection and a BVDV-2 infection.

An embodiment of the invention relates to a BoHV-1 recombinant vector above for use as a vaccine.

An embodiment of the invention relates to a BoHV-1 recombinant vector above for use as a vaccine.

An embodiment of the invention relates to a BoHV-1 recombinant vector above for use as a vaccine, wherein the truncation of gG disrupts the chemokine binding ability of glycoprotein gD. preferably the truncation includes a deletion of amino-terminal amino acid residues 1-67, most preferably the truncated sequence of the glycoprotein Gg is replaced by a sequence having at least 90%, at least 95% or 100% sequence identity with the sequence SEQ ID NO:3.

An embodiment of the invention relates to a BoHV-1 recombinant vector above for use in the prevention and/or the treatment of viral diseases, preferably viral respiratory diseases, most preferably bovine viral respiratory diseases induced by at least one of the viruses selected from BVDV-1, BVDV-2, BoHV-1, BRSV and RVFV.

An embodiment of the invention relates to a BoHV-1 recombinant vector above for use in the prevention and/or the treatment of a bovine viral respiratory diseases induced by at least one of the viruses selected from BVDV-1, BVDV-2, BoHV-1, BRSV and RVFV, wherein the BoHV-1 recombinant vector comprises a sequence having at least 90%, at least 95% or 100% sequence identity with a sequence selected from SEQ ID NO: 7 in combination with SEQ ID NO: 8 or SEQ ID NO: 10 or SEQ ID NO: 7 in combination with SEQ ID NO: 10.

Previously, to improve the gEA marker vaccine's vaccine efficacy, the inventors engineered a BoHV-1 triple gene-mutated virus (BoHV-1 TMV). (See U.S. application Ser. No. 15/780,900 In the BoHV-1 TMV: i) the coding sequences of the UL49.5 ectodomain residues 30-32 and the entire cytoplasmic tail residues 80-96 were deleted, and ii) the coding sequences for the gE cytoplasmic tail residues 452-575 (372 bp), the gE-Us9 intergenic region and the entire Us9 ORF (541 bp) were deleted [30]. Vaccine efficacy of the BoHV-1 TMV was compared with that of a gEΔ virus against a virulent BoHV-1 wt challenge and determined that its protective efficacy was significantly better than the gEΔ vaccine while retaining the safety and serological marker properties of the gEΔ virus [31]. Results showed that both after vaccination and challenge, BoHV-1 TMV generated a considerably better cellular immune response in calves. After the BoHV-1 wt challenge and compared with that of the sham- and gEΔ-vaccinated calves, BoHV-1 TMV-vaccinated calves; i) had more rapid and significant increases in neutralizing antibody titers and ii) had a markedly reduced and shorter duration of nasal virus shedding [30].

The present invention was initially developed to use the BoHV-1 TMV as a delivery vector for a BVDV subunit vaccine. A construct designated BoHV-1 TMV-BVDV.E21, encoding a BVDV-1 envelope glycoprotein E2 was generated. However, BoHV-1 TMV-BVDV.E21 was less effective than a commercial MLV against a virulent BVDV-1 challenge, and thus, it needed improvement. Accordingly, the chemokine binding, gG envelope protein was also deleted. In the resulting quadruple gene-deleted BoHV-1 (BoHV-1 QMV), the gG-dependent blockade of chemokine signaling for immune evasion was eliminated. The modified BoHV-1 QMV vector was used to generate novel constructs whereby the genes encoding BVDV.2 E2 and chimeric Erns bovine granulocyte-macrophage colony-stimulating factor (GM-CSF) fusion (Erns-GMCSF) proteins were inserted in the gE CT-Us9 and gG deletion loci, respectively. GM-CSF is known to enhance both humoral and cellular immune responses in viral vaccines [32]. The BVDV-2 E2 antigen is expressed using an expression cassette as defined in SEQ ID NO: 7 (FIGS. 2B and 3). The chimeric Erns and GMCSF gene as disclosed in SEQ ID NO: 8 is inserted into the modified BoHV-1 QMV vector by homologous recombination thanks to the flanking sequences located upstream and downstream of the expression cassette encoding for the chimeric Erns-GMCSF proteins (FIGS. 2E-2F and 4A-4B).

The results presented here demonstrate that the present invention expressing the BVDV-2 E2 and Erns -GMCSF (QMV-E2/Erns-GMCSF), hereafter designated as QMV-BVD2*, is a safe and effective vaccine for the protection of calves against BVDV-2. The QMV-BVD2* prototype subunit vaccine induced the BoHV-1 and BVDV-2 neutralizing antibody responses along with BVDV-1 and -2 cross-reactive cellular immune responses. Moreover, after a virulent BVDV-2 challenge, the QMV-BVD2* prototype subunit vaccine conferred a more rapid recall BVDV-2-specific neutralizing antibody response and a considerably better recall BVDV types 1 and 2-cross protective cellular immune responses than that of a commercial trivalent (BoHV-1, BVDV-1 and -2 strains) MLV (Zoetis Bovi-shield Gold 3).

### BRIEF DESCRIPTION OF THE DRAWINGS

FIG. 1 is a schematic showing the genomic configuration of BoHV-1 TMV constructed previously and the strategy of BoHV-1 gG gene deletion to generate a BoHV-1 QMV vaccine vector.

FIG. 2 is a schematic showing the construction strategy of chimeric BVD2 E2 and Erns-GMCSF insertion in the gE CT-Us9 and gG deletion loci respectively of BoHV-1 QMV genome to generate BoHV-1 QMV-BVD2 E2-Erns-GMCSF virus designated as QMV-BVD2*. SEQ ID NO: 2 and SEQ ID NO: 3 are disclosed, respectively in order of appearance.

FIG. 3 shows the nucleotide sequence of chimeric BVDV-2 E2 gene cassette defined as SEQ ID NO: 7.

FIG. 4A and 4B show the nucleotide sequence of chimeric BVDV-2 Erns-GMCSF-Flag gene insertion plasmid (pBVD2-E2.INS) defined as SEQ ID NO: 8.

FIGS. 5A, 5B and 5C shows the codon-optimized nucleotide sequence of RVFV chimeric Gn-GMCSF-Flag-Peptide 2A-Gc-V5 cassette defined as SEQ ID NO: 10.

FIGS. 6A and 6B shows the amino acid sequence of the RVFV chimeric Gn-GMCSF-polypeptide defined as SEQ ID NO: 11 and the chimeric GSC+Peptide 2A-Gc polypeptide defined as SEQ ID NO: 12.

FIG. 7 is a schematic showing RVFV chimeric Gn-GMCSF-Flag-Peptide 2A-Gc-V5 cassette incorporated into the gGΔ locus of BoHV-1 QMV resulting in QMV-RVFV*.

FIG. 8 is a schematic showing the vaccination, sample collection, challenge, and euthanasia scheme for the animal experiment.

FIG. 9 shows In vitro characterization of QMV-BVD2* by Immunoblot (A), plaque size analysis (B), and one-step growth analysis (C).

FIG. 10 shows the immunoblot analysis of QMV-RVFV* vaccine virus expressing chimeric RVFV Gn-GMCSF-Flag (left panel) and Gc-V5 (right panel) proteins.

FIG. 11 shows the subcellular localization by indirect immunofluorescence for chimeric RVFV Gn-GMCSF (B/C/D) and Gc (A/C/D) proteins expression in the QMV-RVFV*-infected MDBK cells (magnification 10×).

FIG. 12 shows serum neutralizing antibody titers after vaccination and challenge by standard plaque reduction assay against BoHV-1 (top panel) and BVDV-2 (bottom panel).

FIG. 13 is a table providing serum neutralizing antibody response against BVDV 125 post vaccination and post-challenge in individual animal.

FIG. 14 shows pre-vaccination, post-vaccination, and post-challenge BVDV-1 (A) and BVDV-2 (B) strain-specific INF-γ cellular response by ELISPOT assay.

FIG. 15 shows post-vaccination and post-challenge BVDV-1 (A) and BVDV-2 (B) strain-specific proliferation of the PBMCs by cell proliferation assay.

FIG. 16 shows leukopenia after challenge in sham-, “bovi”- and QMV-BVD2*-vaccinated animals.

FIG. 17 is a table showing leukocyte counts in individual animals pre- and post-challenge.

FIG. 18 shows BVDV nasal virus shedding (A) and viremia (B) following challenge with BVDV2 890 strain.

FIG. 19 is a table providing nasal virus shedding of BVDV in individual animal post-challenge.

FIG. 20 is a table providing viremia data in individual animal post-challenge.

FIG. 21 shows assessment of rectal temperature (A) and clinical score (B) of sham-, “bovi”- and QMV-BVD2*-immunized animals following vaccination and challenge.

FIG. 22 is a table providing rectal temperatures in individual animal before and post-challenge.

FIG. 23 is a table detailing clinical scoring scheme.

FIG. 24 is a table providing clinical score in individual animal post-challenge.

FIG. 25 shows gross pathology of lung in sham- and 2 “bovi”-vaccinated calves.

FIGS. 26A and 26B show histopathology of representative sections of lung tissues showing lesions from sham-vaccinated animals at different magnifications.

FIGS. 27A and 27B show histopathology of representative sections of lung tissues showing lesions from “bovi”-vaccinated animals at different magnifications.

FIGS. 28A and 28B show histopathology of representative sections of lung tissues showing lesions from QMV-BVD2*-vaccinated animals at different magnifications.

FIG. 29 shows means histopathology lesions across treatment groups by areas of lung tissues.

FIG. 30 is a table providing lung lesion score for individual animal.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be use and other methods developed without departing from the broad concepts of the current invention. All references cited anywhere in this specification, including the Background and Detailed Description sections, are incorporated by reference as if each had been individually incorporated.

Definitions are included herein for the purpose of understanding the present subject matter and the appended claims. The abbreviations used herein have their conventional meanings within the chemical and biological arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The present description identifies certain nucleotide and amino acid sequences (polynucleotides and polypeptides) as part of the invention. It is to be understood that the specifically identified sequences adequately describe other sequences that contain less than 100% sequence identity but to the identified sequences that provide the same function. For example, a nucleotide sequence may have 90% sequence identity or 95% sequence identity with a polynucleotide specifically disclosed herein and still encode for an entirely equivalent or functionally equivalent polypeptide. Similarly, a polypeptide may contain less than 100% sequence identity to a polypeptide specifically identified herein and provide the same function. For example, a polypeptide may have 90% sequence identity or 95% sequence identity with a polypeptide specifically disclosed herein and still retain the same or sufficiently similar activity or functionality as the specifically identified polypeptide.

As used throughout, the term “chimeric gene” refers to a hybrid gene having a nucleotide sequence comprising at least two partial or complete sequences derived from, obtained from, or isolated from different genes that are not naturally adjoined. A chimeric protein or chimeric polypeptide is the functional product of a chimeric gene. Chimeric gene can further be modified by mutation, deletion, insertion or substitution of heterologous sequences, or by any means available using recombinant DNA technology.

Throughout, the terms “BHV-1” and “BoHV-1” refer to the bovine alpha-herpesvirus type 1 and can be used interchangeably.

In some embodiments, a “recombinant vector” as used herein refers to a genetic material, for example a virus or a plasmid, used as a vehicle to artificially carry foreign genetic material into a host cell where it can be replicated and/or expressed. Such vehicle has been genetically engineered to produce new genetic combination.

As used throughout, the term “mutant virus” refers to a virus which has been genetically engineered by deletion, mutation or truncation of genetic sequences and/or by subsequent insertion or substitution of heterologous genetic sequences. When used as vaccine, such mutant virus becomes less pathogenic, while still being able to elicit robust immune responses in a host.

Given that the engineered quadruple mutant virus described herein is used as a recombinant vector to carry and express protective viral antigens, both terms “mutant virus” and “recombinant vector” are used interchangeably throughout.

As used throughout, the term “live-attenuated” or “modified live” refer to a live organism such as a virus which has been weakened so that it is not virulent but can still induce protective immune responses in a host.

As used throughout, the term “heterologous” refers to any material which originates from a different viral strain, a virus of a different type, a bacteria, a mammal or any species different from that of the BoHV-1

As used throughout, the term “cytopathic” refers to a virus which causes the death of the infected cells, whereas the term “non-cytopathic” refers to a virus which propagate without killing the infected cell.

As used throughout, the terms “bovi” or “bovi vaccine” refer to the Bovi-Shield Gold® 3, a commercially available modified live virus (MLV), which provides protection against three important bovine respiratory disease conditions, i.e., BoHV-1, BVDV-1 and BVDV-2.

As used throughout, the abbreviations “dpv” and “dpc” refer to day post-vaccination and day post-challenge, respectively.

Current vaccination practices against the viruses causing BRDC include trivalent attenuated, BoHV-1, BVDV-1 and -2 live vaccines. While these vaccines protect against the severity of BoHV-1 and BVDV infections, these vaccines were linked to outbreaks of abortion (BoHV-1) in dairy cattle industries, respiratory diseases (BoHV-1 and BVDV) in the beef and dairy cattle industries, and persistent infections (BVDV) in dairy cattle industries. In several cases, the causal agent(s) could be traced back to the vaccine strain of BoHV-1 used in the polyvalent vaccine because the traditional BoHV-1 MLV vaccine virus establishes latency in the TG, reactivates with stress and can be shed in nasal secretions. Therefore, only the gE-deleted BoHV-1 marker vaccine is allowed in several EU countries for vaccination against BoHV-1. The BoHV-1 gE-deleted marker vaccine is distinguishable from the BoHV-1 MLV strains serologically. Under field conditions, the gE marker vaccine virus in most cases was not shed from the nose of vaccinated animals following reactivation from the latency. However, a low-level gE marker virus shedding occurred in some instances of latency-reactivation (http://ec.europa.eu/food/fs/sc/scah/out49_en.pdf)[29].

The live attenuated BVDV strains used in the multivalent bovine respiratory disease vaccines are suspected in BVDV-associated problems in the cattle industry because of its RNA genome's inherent ability to mutate under the field conditions. Additionally, like the wild type (wt) VDV, the vaccine virus also causes immunosuppression and vertical transmission in pregnant cows and persistent infection of calves [19, 43-46]. Recently, the single Npro and double Npro-Erns live BVDV mutants were also developed to avoid the traditional BVDV MLV vaccine-associated problems. However, both the mutant viruses can cross the placental barrier and established persistent infection [19, 47]. Therefore, traditional MLV and genetically engineered BVDV vaccines are not allowed in many EU countries or discouraged. Instead, eradication of BVDV by i) testing and identifying newborn calves for persistent BVDV infection, ii) removing PI calves, and iii) taking hygienic, and biosecurity control measures have been implemented. However, this latter approach renders the naïve cattle population vulnerable to severe and widespread BVDV infection if the virus is introduced into the cattle population.

In some embodiments, a BoHV-1 quadruple mutant virus (BoHV-1 QMV) refers to an engineered virus which lacks the BoHV-1 UL49.5 Ectodomain residues (30-32) plus the CT residues (80-96), the entire gE CT and Us9, and gG. A portion of the nucleotide sequence of the glycoprotein gG was deleted in order to disrupt the functions of gG, in particular its binding ability to chemokines. In an embodiment, the deletion encompassed the sequence encoding for the amino terminal sequence of gG, preferably the N-terminal amino acid residues 1 to 67. In an embodiment, a short sequence defined as SEQ ID NO: 3 was inserted in the gG deletion locus.

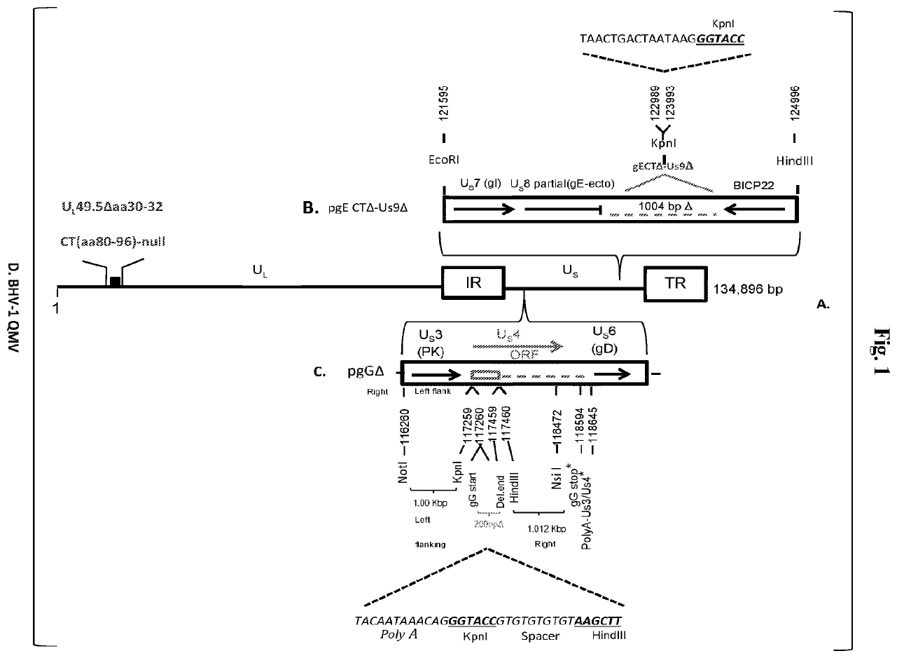

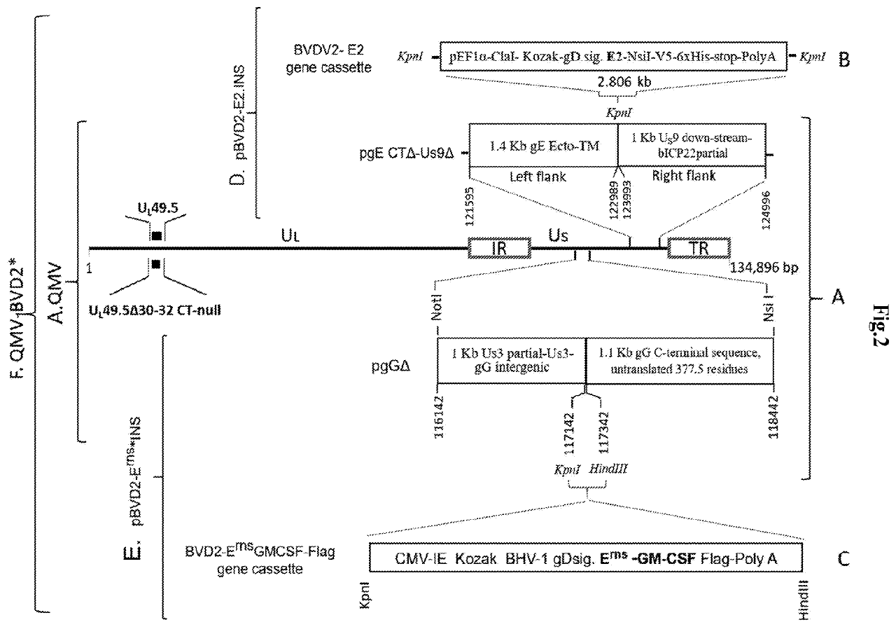

Some embodiments of the invention further include the chimeric BVDV-2 E2 and Erns-GMCSF genes which are inserted in the gE CT-Us9 (FIG. 2B) and gG deletion (FIG. 2C) loci, respectively, thus resulting in QMV-BVD2* recombinant vector (FIG. 2F). The gE CT-Us9 locus comprises a short 21 nucleotide sequence with a KpnI restriction site as defined in SEQ ID NO: 2 (FIG. 1B). In the gG deletion locus, a short 34 nucleotide sequence comprising the KpnI and HindIII restriction sites as defined in SEQ ID NO: 3 was inserted (FIG. 1D).

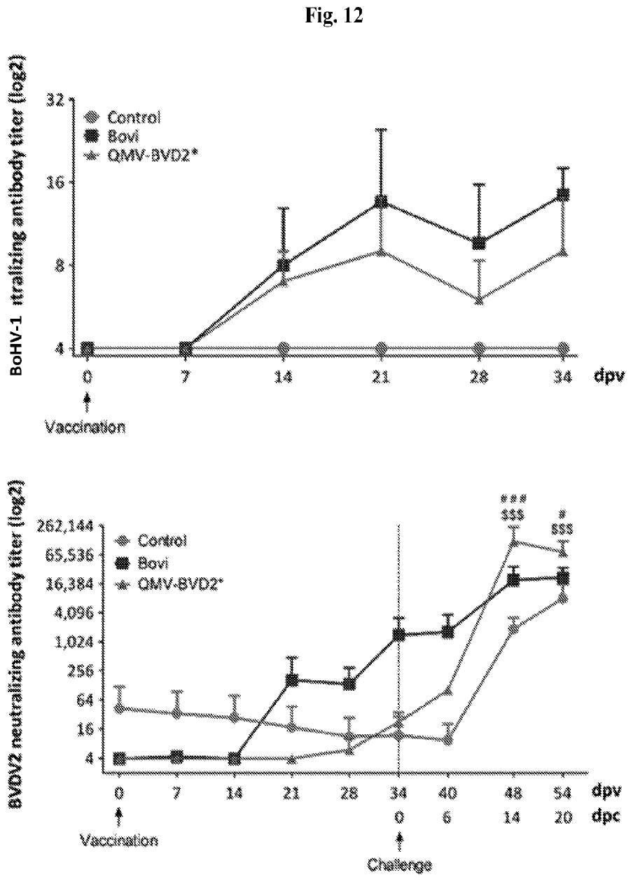

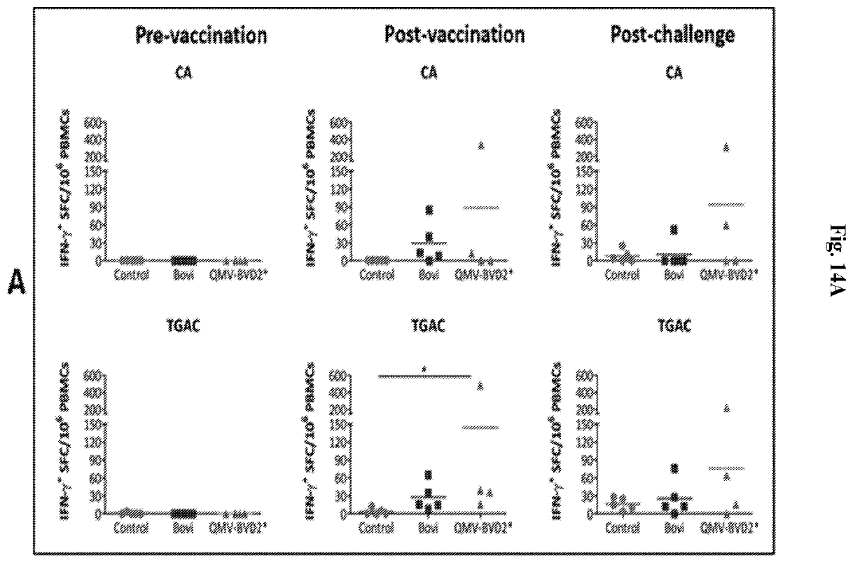

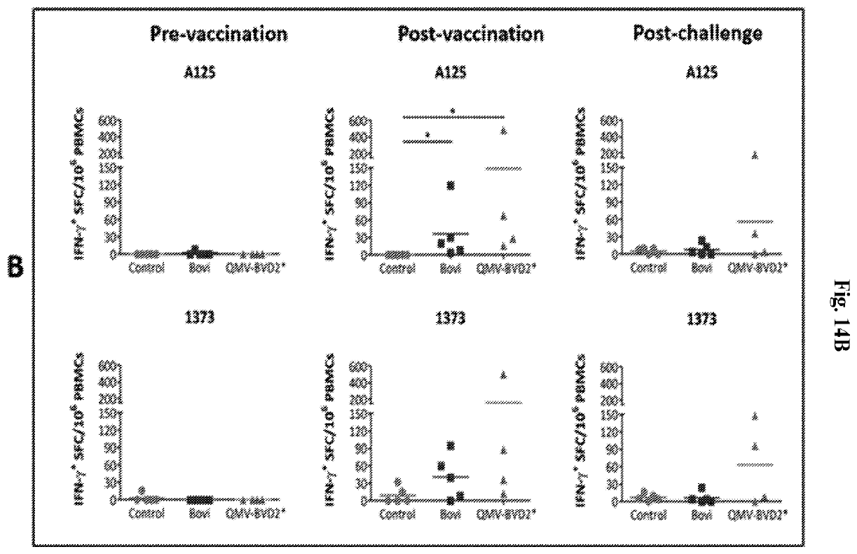

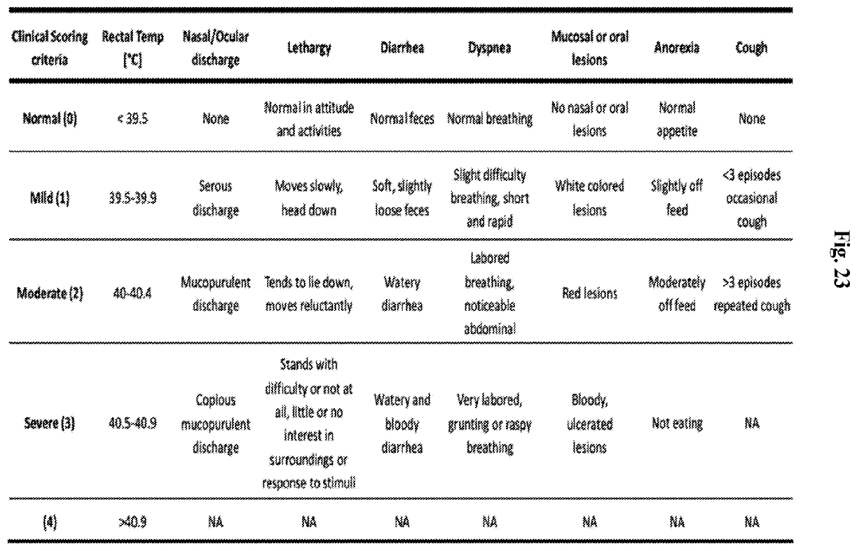

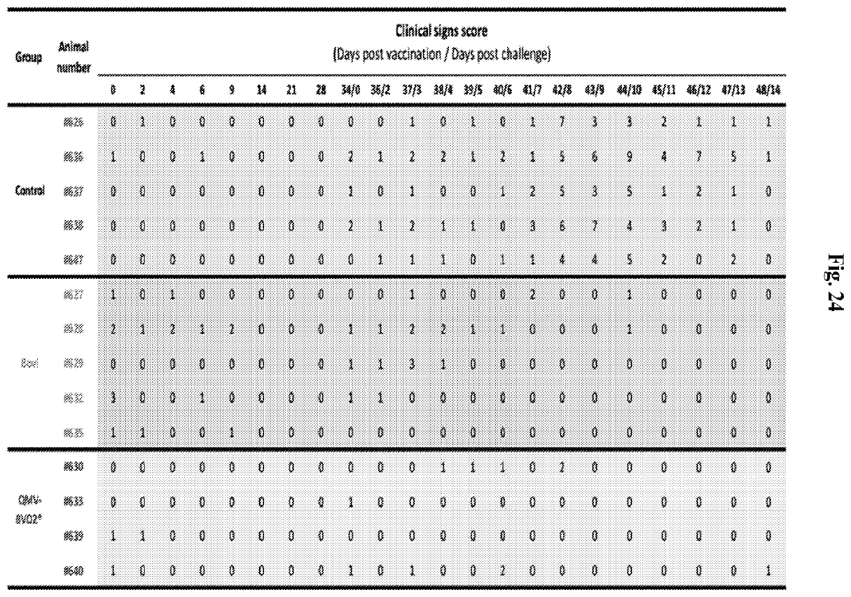

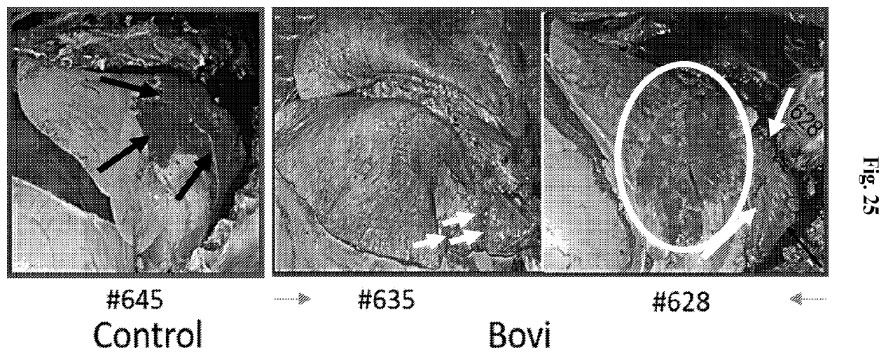

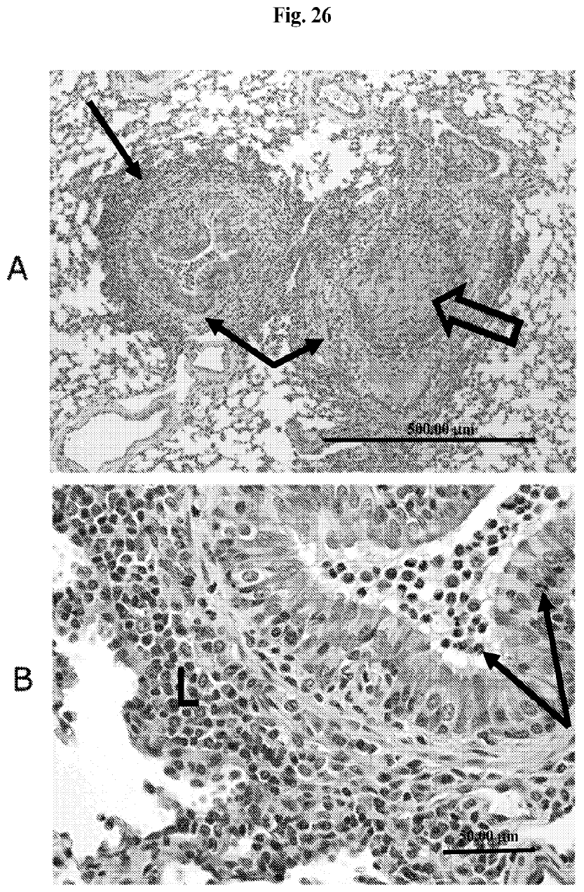

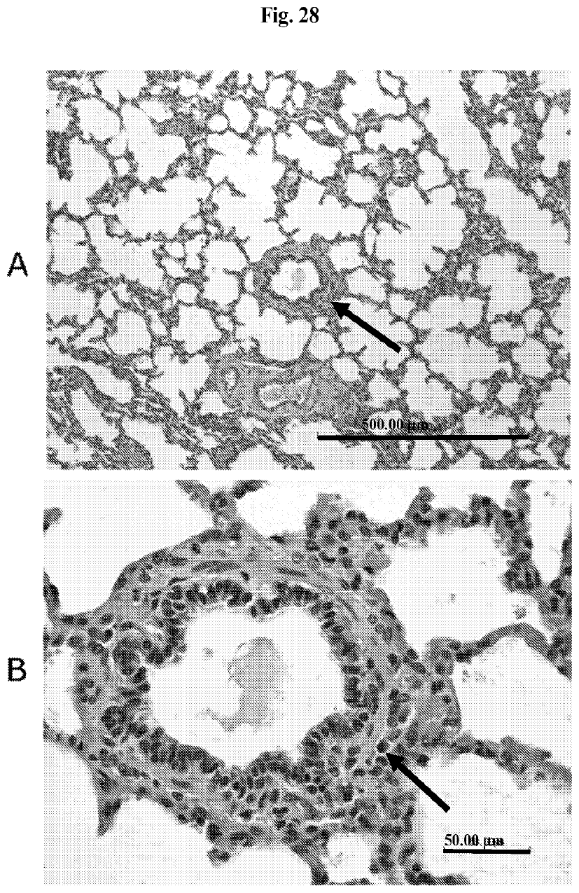

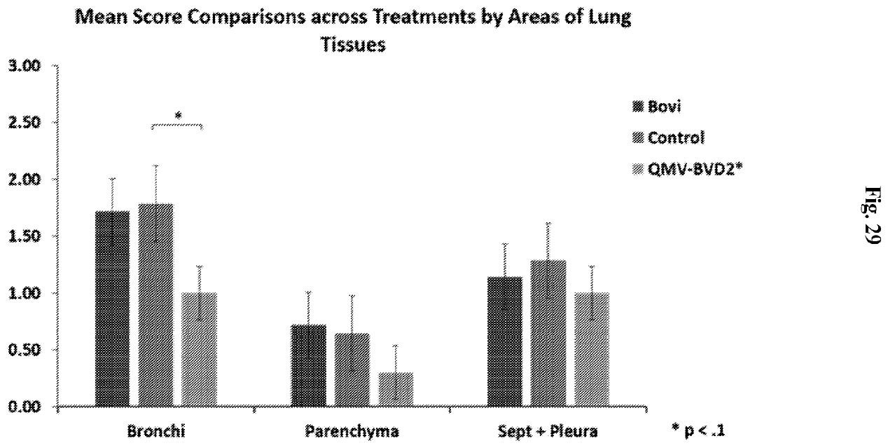

The QMV-BVD2* vaccine elicited higher cross-reactive IFN-γ and proliferation responses in the vaccinated calves against BVDV-1 and -2 before and after the virulent BVDV-2 challenge when compared with the “Bovi”-vaccinated group (FIGS. 14 and 15). The QMV-BVD2* vaccine also induced BVDV-2-specific seroconversion at 34 dpv, but the “Bovi” generated a 50-fold higher BVDV-2 serum-neutralizing titer by that time. Nevertheless, QMV-BVD2* vaccinated calves had a better recall neutralizing antibody response after the challenge (6 dpc), and the BVDV-2-specific SN titer increased four-fold in the QMV-BVD2*-vaccinated calves (17 to 64). In contrast, the SN titers in the “Bovi” group remained the same (878 on 0 dpc and 885 on 6dpc). Notably, by 14dpc, the SN titer in the QMV-BVD2* sky-rocketed 5,500-fold relative to that on the day of the challenge, whereas the corresponding increase of SN titer in the “Bovi” group was 19-fold (FIG. 12). Taken together, upon BVDV-2 challenge, QMV-BVD2* vaccinated calves had stronger and higher levels of BVDV cross-reactive (types 1 and 2) T cells as well as BVDV-2-specific recall SN neutralizing responses compared with that of “Bovi”. Most notably, the calves in both the control-unvaccinated and the “Bovi” vaccinated groups had visible gross lung-lesions (FIG. 25). Consistent with this finding, the animals in the “Bovi” and sham-vaccinated groups had also lung histopathological lesions (FIG. 26A, 26B, 27A and 27B); however, the QMV-BVD2* vaccinated calves had either mild or no lesion (FIG. 28A and 28B). Therefore, the QMV-BVD2* primes and induces a better BVDV-specific memory T cell response than that of “Bovi” (MLV), suggesting a long-lasting protection as it is often the case for vaccines that elicit strong cellular and humoral responses

Consistent with this assumption, previous attempts to use BoHV-1 vectored BVDV subunit E2 vaccines were not adequately protective even though they induced BVDV-specific neutralizing antibody response [48, 49]. Notably, these BHV-1 vectors still had the two immunosuppressive genes, UL49.5 and gG, intact in their genome. Also, in those instances, only the BVDV E2 was used as a subunit antigen. In contrast, BVDV2 Erns fused with the bovine GM-CSF chimeric protein is additionally used as a second subunit antigen.

Taken together, deleting both the immunosuppressive BoHV-1 genes in the vaccine vector combined with the inclusion of GM-CSF together with Erns most likely contributes towards improved cellular and memory neutralizing antibody responses against BVDV. Remarkably, even though the subunit antigens expressed by the QMV-BVD2* were type 2 BVDV-specific, the cellular immune response induced by the prototype vaccine was reactive against both BVDV-1 and -2. Earlier, it was also determined that the BoHV-1 TMV was equally attenuated as a gE-deleted virus but induced a better protective immune response against the virulent BoHV-1 challenge compared with the gE-deleted virus with respect to both cellular immune response and neutralizing antibody responses. In the case of QMV-BVD2*, in which the BoHV-1 gG gene was additionally deleted, the efficiency of virus replication in the nasal mucosa was reduced slightly compared with that of TMV (Chowdhury et al., 2014). Nevertheless, BoHV-1 QMV induced slightly higher BoHV-1-specific neutralizing antibody response compared with that of BoHV-1 TMV [30]. However, based on its comparable neutralizing antibody response to that of “Bovi”-vaccinated animals, the QMV-BVD2* is equally or better protective against BoHV-1 than the BHV-1 TMV.

Taken together, this demonstrates that the QMV-BVD2* vaccine is similarly or slightly better protective against BoHV-1, BVDV-1, and BVDV-2 compared with that of “Bovi” vaccine. By using the QMV-BVD2* vaccine, comparable or equal protection against the three viruses is obtained while avoiding the MLV BoHV-1 and BVDV vaccines associated problems in the field, for example, shedding after latency-reactivation (for BoHV-1 MLV), high mutation rate, immunosuppression, and vertical transmission (for BVDV).

From a manufacturing point of view, the vaccine is cost-effective. Rather than growing three different viruses (BoHV-1, BVDV-types 1 and 2) to formulate the vaccine, only one QMV-BVD2* is needed. Additionally, QMV-BVD2* grows at a much higher titer in MDBK cells compared with that of BVDV, thus providing BVDV protective antigens. Furthermore, based on the gE CT-based marker assay [31], the QMV-BVD2*-vaccinated animals can be distinguished from the wt BoHV-1-infected animals in the field. A BVDV NS3-based blocking ELISA test which is commercially available (BIO K 230; Biox Diagnostics S. A., Rocheforte, Belgium) and could be used to distinguish QMV-BVD2*-vaccinated animals from BVDV-infected animals based on the NS3 serological marker. Therefore, QMV-BVD2* will fulfill the DIVA (Differentiation of Infected and Vaccinated Animals) property against both BoHV-1 and BVDV to distinguish the vaccinated animals from the infected animals under field conditions.

RVFV genome is segmented and consists of L (6404 nt), M (3885 nt) and S (1690 nt) segments. The middle (M) RNA segment of the RVFV genome encodes a 78 kD accessory protein, the viral envelope glycoproteins Gn and Gc, and a nonstructural protein NSm. The Gn and Gc contain the N terminal residues 154-690 and the C-terminal residues 691-1206 of the M segment, respectively. The Gn (54 kD) and Gc (59 kD) are produced after cleavage of the polyprotein (encoded by the M segment) by host proteases and form a heterodimer in the endoplasmic reticulum (ER). The heterodimerization is required for the transport and maturation of Gc in the Golgi compartment. The GnGc heterodimer in the virion envelope facilitates virus binding and entry into the host cells.

To date, a vaccine vector lacking multiple properties, i.e., virulence, immuno-suppressive and recurrent nasal virus shedding upon reactivation from latency has not been developed and tested for its efficacy as a potential vaccine vector against RVFV. Therefore, developing the QMV-RVFV* vectored subunit vaccine against RVFV is needed to control a potential RVFV outbreak in livestock, reduce the mortality and morbidity associated with RVFV in livestock, and in turn hamper the risk of RVFV transmission from sheep and cattle to human. More specifically, it is important to develop a QMV-RVFV* vaccine expressing the Gc and Gn epitopes which can induce both humoral and cellular immune response in livestock, for a robust and durable protection against the viral disease.

The invention is described herein by the following representative non-limiting example intended only to teach those skilled in the art the best way known to the inventors to make and use the invention. Nothing in this example or specification should be considered as limiting the scope of the present invention. The specific embodiments of the invention described may be modified or varied, without departing from the invention, as appreciated by those skilled in the art in light of the above teachings. It is therefore to be understood that, within the scope of the claims and their equivalents, the invention may be practiced otherwise than as specifically described.

### EXAMPLE

### 2. Materials and Methods

### 2.1. Cells

The Madin Darby bovine kidney (MDBK) cell line was maintained in Dulbecco's modified Eagles medium (DMEM #10-017-CV, Corning, MA, USA) supplemented with 10% heat-inactivated fetal bovine serum (FBS; EquaFETAL, Atlas Biologicals, CO, USA) and 1× antibiotic/antimycotic solution (cat #30-004-CI; Corning).

### 2.2. Viruses

BoHV-1 wild type Cooper (Colorado-1) strain was obtained from the American Type Culture Collection (ATCC # VR-864), and low passage viral stocks were maintained at −80° C. BoHV-1 TMV was generated previously [30]. The cytopathic (cp) BVDV-1a Singer strain was received from LSU Louisiana Animal Disease Diagnostic Laboratory (LAADL). BVDV-1b cp strain TGAC was received from Dr. C. Chase from South Dakota State University [18]. BVDV-2a (cp) strain 125 was kindly provided by Dr. Clayton Keling, the University of Nebraska, at Lincoln, Nebraska. BVDV-1b non-cytopathic (ncp) strain CA04011866a (designated hereafter as CA), and ncp BVDV-2a strains 890 and 1373 were obtained from USDA/APHIS, Aimes, Iowa.

### 2.3 Antibodies

BVDV types 1 and 2, E2-specific monoclonal antibody (mAb; #348) and BVDV-2 E2-specific mAb (#BA-2) were from VMRD® (WA, USA). Anti-Flag-specific mAbs (#F1804 or #F7425) was from Sigma-Aldrich (MO, USA). Anti-VS-specific mAb (Ab #R96025) was from Thermo Fisher. Donkey anti-mouse highly cross-absorbed secondary antibody conjugated, Alexa Fluor 488 (#A-21202) and the Alexa Fluor 647 donkey anti-rabbit IgG were from Invitrogen (CA, USA).

### 2.4. Virus Titrations

Virus titration, in the cases of BoHV-1 and cytopathic BVDV-2 strain 125 (125) was performed by plaque assay. Each viral stock solution was serially diluted ten-fold in DMEM supplemented with 5% FBS and 1× antibiotic/antimycotic solution. 200 μl of each virus-dilution was applied in duplicate onto the wells of 24-well cell culture plates over confluent MDBK cells. The plates were incubated for 2 h at 37° C. in a CO2 incubator before cells were overlaid with 1.6% carboxyl methylcellulose (CMC-high viscosity, Sigma-Aldrich, USA, #C5013) in DMEM. After 48 h (BoHV-1) and 72 h (BVDV-2), cells were fixed with 10% formalin solution for 1 h at room temperature (RT) and stained with 0.35% crystal violet. Plaques were counted under a surgical microscope. Virus titer was expressed as plaque-forming units (PFUs)/ml by using the following calculation: Reciprocal of the highest virus dilution x average number of plaques (5-20 plaques) counted in the two wells×5. The viral plaque assay of BVDV-2 (ncp) strain 890 was performed similarly as above (for 125), but the cells were fixed at RT for 20 min (3% paraformaldehyde solution in PBS), and the viral plaques were

visualized by immunofluorescence assay using the BVDV-specific (both types 1 and 2) mAb #348 (VMRD®).

### 2.5. Construction of BHV-1 QMV Vector Virus

BoHV-1 TMV was constructed earlier, in which i) UL49.5 residues 30-32 and CT residues 80-96 were deleted and ii) the entire gE CT-Us9 coding regions were deleted (FIG. 1A-1C). FIG. 1A discloses the BoHV-1 U_{L} 49.5Δ30-32 CT-null virus backbone having an unique long region (U_{L}), an unique short region (U_{S}), an internal repeat region (I_{R}), and a terminal repeat region (T_{R}) [14]. FIG. 1B discloses the plasmid construct pgE CTΔ-Us9Δ used to introduce CTΔ-Us9Δ in the BoHV-1 U_{L} 49.5Δ30-32 CT-null virus backbone (A) to generate a BoHV-1 triple mutant virus (BoHV-1 TMV) [30]. FIG. 1C discloses a short nucleotide sequence (SEQ ID NO: 2) comprising a KpnI restriction site, which can be used as an insertion site to insert an expression cassette encoding an antigen, such as the BVD2 E2-expression cassette as defined in SEQ ID NO: 7. FIG. 1C provides the genomic configuration of BoHV-1 TMV. To further improve the immunogenicity of BoHV-1 TMV, the gG gene in the BoHV-1 TMV genome was deleted/null-mutated and a BoHV-1 QMV generated. The BoHV-1 gG (Us4) is flanked by Us3 and Us6 (gD) genes, on the left and right, respectively (FIG. 1D). The genomic organization of BoHV-1 QMV is shown in FIG. 1E and its GenBank accession number is JX898220.

A viable gG ORF-deleted virus could not be isolated when the entire gG ORF coding region was deleted. It was suspected that the putative gD gene promoter sequence (an essential viral gene) might be partially overlapping with the gG ORF sequence's carboxy end. Alternatively, it could be that the deletion might have affected the shared Us3/Us4 Poly A site (FIG. 1D), situated down-stream of the gG stop codon (nt 118640-118645). Therefore, a strategy was developed i) to delete only the coding region of amino-terminal 67 amino acids, including the start codon of gG; and ii) to incorporate a synthetic Poly A replacement sequence in the deletion site to compensate for the authentic Us3 Poly-A, and the KpnI and HindIII restriction sites as defined in SEQ ID NO:3. The 34 nucleotide sequence (SEQ ID NO:3) can be used to insert an expression cassette encoding one or more heterologous antigens such as the BVDV-2 Erns-GMCSF chimeric gene expression cassette2055 bp long sequence, containing 5′-3′ was designed as follows: The 1000 bp US3 (partial) and US3-gG intergenic sequence (nt 116260-117259) with a Notl restriction site at the 5′ end, followed by 34 bp sequence as defined in SEQ ID NO:3 and containing 5′-3′, a replacement Us3 PolyA sequence as it appears in the BoHV-1 genomic sequence with its 3 bp flanking sequence on either side (nt 1186637 to 118648), a KpnI restriction site, a 10 bp spacer sequence and a HindIII restriction site (FIGS. 1D and 2A). The HindIII restriction site is flanked on the right by the 1012 bp carboxy-terminal gG ORF sequence (nt 117460-118472) followed by a NsiI restriction site at the 3′ end (FIGS. 1D and 2A). According to this configuration of the designed 2055 bp long sequence, a 200 bp gG ORF sequence coding for the gG amino-terminal 67 amino acids, including the start codon (nt 117260- 117459) were deleted, and the downstream gG amino acid residues 68-444 (nt 117460-118620) were not translated. Further, the gG deletion locus was flanked by 1000 bp on the left and 1160 bp BoHV-1 genomic sequences on the right sides, respectively, for homologous recombination into the BoHV-1 genome (FIGS. 1D and 2A). The 2055 bp Notl-Nsi fragment designed above was synthesized and cloned into the corresponding sites of the plasmid pBME-amp. The resulting plasmid clone's integrity, pgGA, was verified by sequencing (Biomatik Corporation, Ontario, Canada). A BoHV-1 QMV was generated by cotransfection/homologous recombination of pgGΔ with the full-length BoHV-1 TMV DNA using Lipofectamine (Invitrogen) as described earlier (FIG. 2). Several putative BoHV-1 QMV recombinants were analyzed by sequencing the genomic region spanning the Us3-gD genes. One of the recombinants was selected for the insertion of the BVDV-2 E2 and Erns-GMCSF chimeric genes.

### 2.6. Incorporation of Chimeric BVDV 2-E2 Gene Cassette in the BoHV-1 QMV Genome to Generate BoHV-1 QMV-BVD2-E2

### 2.6.1. Construction of BVDV-2 E2 Insertion Plasmid

The plasmid pgE CTA-Us9Δ was generated previously (Chowdhury et. Al. 2014). Briefly, it contains a 2400 bp BoHV-1 genomic sequence inserted into the EcoRI-HindIII sites of plasmid pGEM-7Z (FIG. 1B). The EcoRI-HindIII fragment consisted of gE ORF sequence, nt 121595-122989 bp coding for 451 gE amino-terminal, gE Ecto- and gE transmembrane amino acids flanked by EcoRl and KpnI at the 5′ and 3′ ends, respectively, were fused to 1004 nt of partial bICP 22 gene sequence flanked by KpnI and HindIII at the 5′ and 3′ ends respectively (FIG. 1B). In this configuration, a 1004bp BoHV-1 genomic sequence (nt 122989-123993) comprised of gE CT amino acids 452-575 (approx. 372 bp), 106 bp gE-Us9 intergenic region, the entire Us9 ORF coding sequence (435 bp), and 88 bp of the Us9-bICP22 intergenic region were deleted (GenBank accession #JX898220). Further, a 21 bp sequence containing four stop codons and a Poly A signal, as well as a KpnI restriction site as defined in SEQ ID NO: 2, was incorporated in the gE CT-Us9 deletion locus (FIG. 1B). The sequence can be used to insert an expression cassette encoding at least one heterologous antigen as a chimeric protein.

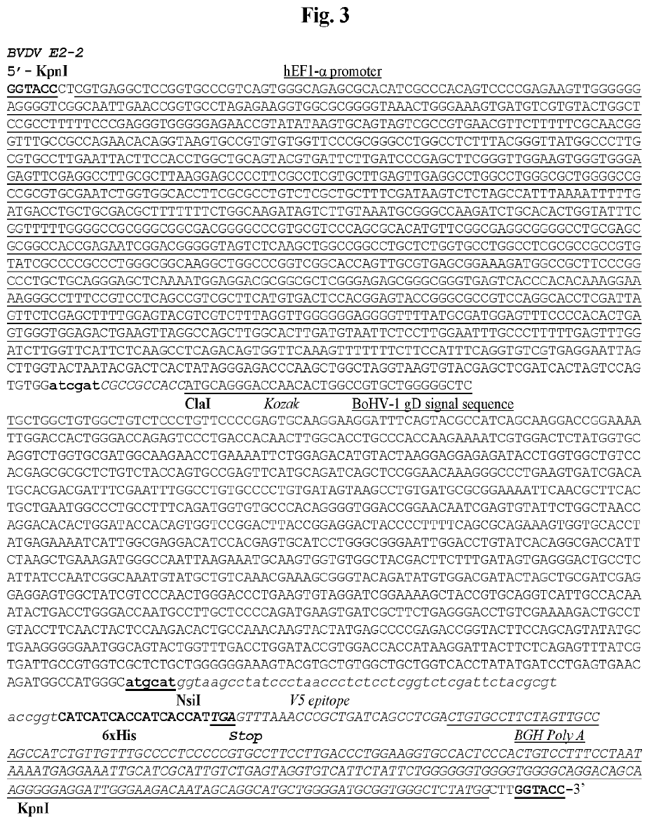

To construct a BVDV-2 E2 insertion plasmid, first, a 2,806 bp BVDV-2 E2 chimeric gene cassette as defined in SEQ ID NO: 7 (pBVD2-E2 gene cassette) was synthesized (Genscript, NJ, USA), which consisted of the following: A 1,286 bp sequence for human elongation factor-1α (EF-1α) promotor flanked by KpnI (5′) and ClaI (3′) restriction sites, followed by a 1,183 bp chimeric sequence containing, the Kozak sequence (SEQ ID NO: 4CGCCGCCACC), BoHV-1 gD signal sequence (nt 118819 to 118875, #JX898220; aa 1-19, GenBank accession #AFB76672.1), and BVDV-2 E2 ORF coding sequence, codon-optimized for Bos Taurus (GenBank accession #AAC72814.1), followed by a 337 bp NsiI-KpnI fragment containing the V5 epitope, 6×His coding sequence (SEQ ID NO: 5), a stop codon (TGA) and bovine growth hormone (BGH) Poly A sequence (FIGS. 2B and 3). The chimeric BVDV-2 E2 gene cassette as defined in SEQ ID NO: 7 was cloned into the KpnI site of pUC 57, and the integrity of the inserted sequence was verified (Genscript).

The 2806 bp chimeric BVDV-2 E2 gene cassette as set forth in SEQ ID NO: 7 (FIGS. 2B and 3) was then recloned into the KpnI site (SEQ ID NO: 2) of the pgE CTA-Us9Δ plasmid clone described above (FIG. 2A). The resulting plasmid clone, pBHV-1 gEACT US9Δ-BVDV2 E2-INS (pBVD2-E2.INS) (FIG. 2D), contains the 2806 bp BVDV-2 E2 chimeric gene flanked by 1400 bp (on the left) and 1000 bp (on the right) BoHV-1 genomic sequences for recombination and incorporation of the BVDV-2 chimeric E2 gene into the gE CT-Us9 deletion site of BoHV-1 genome (FIGS. 2A, B and D). The integrity of the flanking BoHV-1 and the inserted chimeric E2 sequences was verified (Genelab, LSU)

### 2.6.2. Construction of BoHV-1 QMV-BVD2-E2 Virus by Homologous Recombination

To generate a BoHV-1 QMV-E2 virus, linearized pBVD2-E2.INS insertion vector

DNA was transfected with the full-length BoHV-1 QMV genomic DNA. Several putative recombinant viruses were identified by PCR (data not shown). One putative BoHV-1 QMV-E2 recombinant was plaque purified, and the integrity of the flanking BoHV-1 genomic and the chimeric E2 gene sequences were verified by sequencing (Genelab, LSU).

### 2.7 Incorporation of chimeric BVDV-2 Erns-GMCSF-Flag gene cassette in the QMV-BVD2-E2 Genome to Generate QMV-BVD2-E2-Erns-GMCSF-Flag (Designated Hereafter as QMV-BVD2*)

### 2.7.1. Construction of BVDV2-Erns-GMCSF-Flag Insertion Plasmid

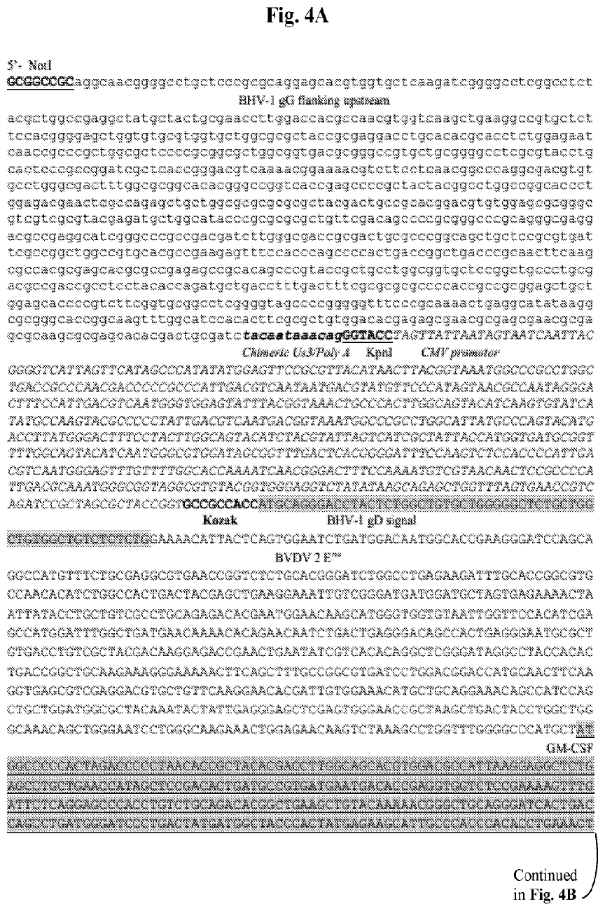

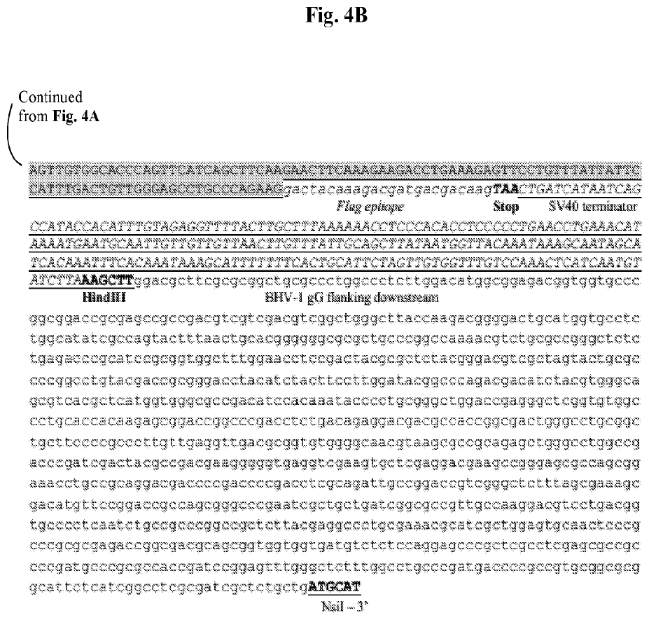

To construct a BVDV-2 Erns-GMCSF-Flag insertion plasmid (pBVD2-Erns*-INS as defined in SEQ ID NO: 8), first a 2,037 bp BVDV-2 Erns-GMCSF-Flag chimeric gene cassette (FIG. 2C and FIGS. 4A-B) was synthsized (Biomatik) as follows: A 605 bp human cytomegalovirus (HCMV) promotor sequence (nt 1 to 605, GenBank #CVU55763) with a KpnI restriction site at the 5′ end, followed by a 1,183 bp nucleotide sequence containing a kozak sequence (SEQ ID NO: 4: CGCCGCCACC), BoHV-1 gD signal sequence (nt 118819 to 118875, #JX898220; aa 1-19, #AFB76672.1), codon-optimized (Bos Taurus) nucleotide sequences for BVDV-2 1373 Erns (aa 271 to 497, #AAD38683) and bovine GM-CSF, lacking the signal sequence (Methionine and residues 18 to 143, #NP776452), the nucleotide sequence coding for a flag tag (SEQ ID NO: 6 GACTACAAAGACGATGA-CGACAAG, variations of the Flag tag can also be used as long as it is recognized by commercially available anti-Flag antibodies), a stop codon (TAA), the simian virus 40 (SV40) termination/polyadenylation (Poly A) site (nt 1411 to 1640, #CVU55763), and the restriction site for HindIII (FIG. 2C). The chimeric BVDV2 Erns-GMCSF-Flag gene cassette (FIG. 4A-B) was cloned into the KpnI-HindIII restriction sites of plasmid pgGA (FIG. 2A), and the integrity of the inserted sequence was verified (Biomatik). In the resulting plasmid clone pBVD2 Erns-GMCSF-Flag insertion plasmid (pBVD2-Erns*-INS), the 2037 bp BVDV-2 Erns-GM-CSF chimeric gene sequence was flanked by 1000 bp on the left and 1160 bp BoHV-1 genomic sequences on the right sides, respectively as set forth in SEQ ID NO: 8 (FIG. 2A, 2E and 4A-B). The integrity of the flanking BoHV-1 and the inserted chimeric Erns-GMCSF sequences was verified (Genelab, LSU).

### 2.7.2. Construction of BoHV-1 QMV-E2-Erns-GMCSF Virus (QMV-BVD2*).

To generate a recombinant QMV-BVD2* vaccine virus, linearized pBVD-2 Erns*INS was cotransfected with full-length QMV-BVDV-2-E2 recombinant genomic DNA, constructed as described above in 2.6.2 (FIG. 2F). Two putative QMV-BVD2* recombinant viruses were plaque purified (3×) and verified by PCR and sequence analyses (Genelab, LSU). Low passage QMV-BVD2* viral stocks were maintained at −80° C.

### 2.8. Mock- and Virus-Infected Cell Lysates, SDS PAGE, and Immunoblotting

For Western Blot analysis of chimeric E2 and Erns-GMCSF expression by QMV-BVD2*, MDBK cells were infected with QMV-BVD2*, BoHV-1 wt, and non-cytopathic (ncp) BVDV-2 890. For QMV-BVD2*- and BoHV-1 wt-infected cell lysates were harvested after 24-36 h when the cytopathic effect was 95-100%. The BVDV-2 890-infected cells were harvested after 5 days. To detect the BVDV E2 and chimeric Erns-GMCSF (fused with Flag tag; FIG. 2C) expressed by the QMV-BVD2* recombinant virus, mock and virus-infected cellular extracts/lysates were prepared and analyzed by 10% SDS-PAGE/Western-immunoblotting with the BVDV-2 E2-specific mAb (vmrd, #BA-2) and anti-Flag-specific mAb (Sigma-Aldrich #F1804) as described earlier [35].

Western Blot analysis of recombinant Gn-GMCSF and Gc expression by QMV-RVFV* were performed following a similar protocol as described above using QMV-RVFV*-infected MDBK cells. Expression was detected using the anti-Flag-specific mAb (Ab #F7425, Sigma-Aldrich) for recombinant Gn-GMCSF-Flag and using anti-VS-specific mAb (Ab #R96025, Thermo fisher) for recombinant Gc-V5.

### 2.9. Comparison of QMV-BVD2 * Growth Characteristics with that of BoHV-1 wt in MDBK Cells

To compare the growth characteristics of QMV-BVD2* with that of BoHV-1 wt, average plaque morphologies and one-step growth curves of QMV-BVD2* and BoHV-1 wt were determined. Two wells of a six-well plate containing a confluent monolayer of MDBK cells were infected with 80-100 PFU of QMV-BVD2* or BoHV-1 wt viruses and overlaid with 1.6% CMC at 2 h post-infection (2 hpi). At 48 hpi, the cells were fixed (10% formaldehyde) and stained with crystal violet. The average plaque size of wt and mutant viruses was determined by measuring approx. 50 randomly selected plaques for each virus under a microscope with a graduated ocular objective, as described earlier (Wei et al., 2011). The one-step virus growth property of the QMV-BVD2* was compared with wt, as described earlier [36]. Virus titers were determined by standard plaque assay as described above in 2.4.

### 2.10. Construction of the BoHV-1 QMV-Gn-GMCSF-Peptide2A-Gc Virus (QMV-RVFV*)

### 2.10.1. Design of the Chimeric Rift Valley Fever (RVFV) Gn-GMCSF-Peptide2A-Gc Chimeric Gene

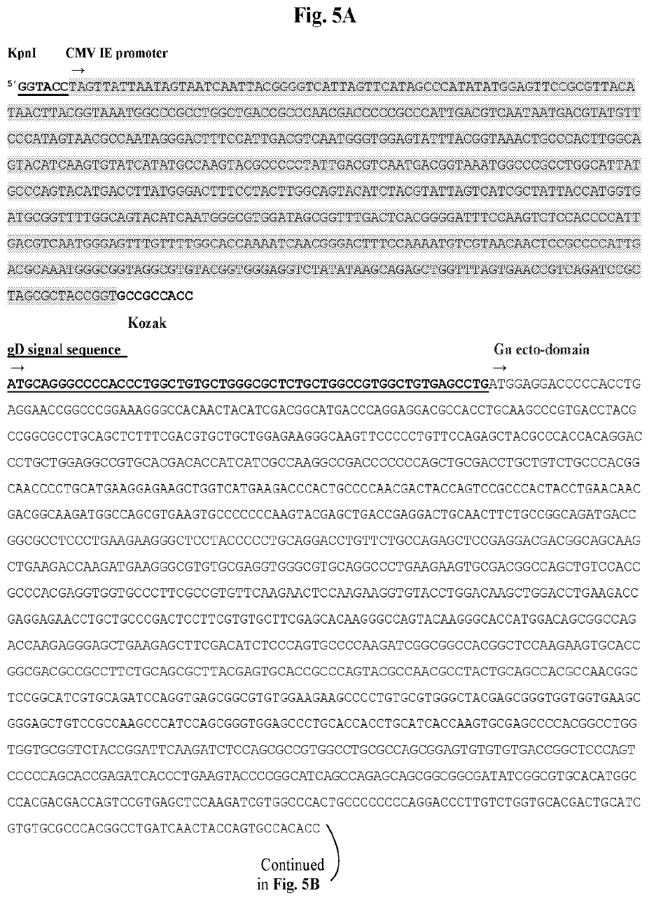

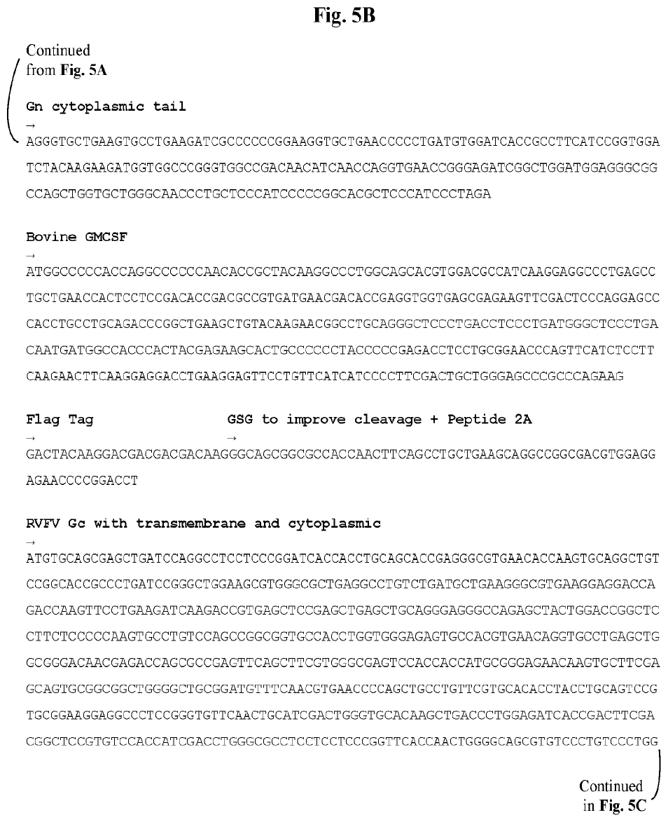

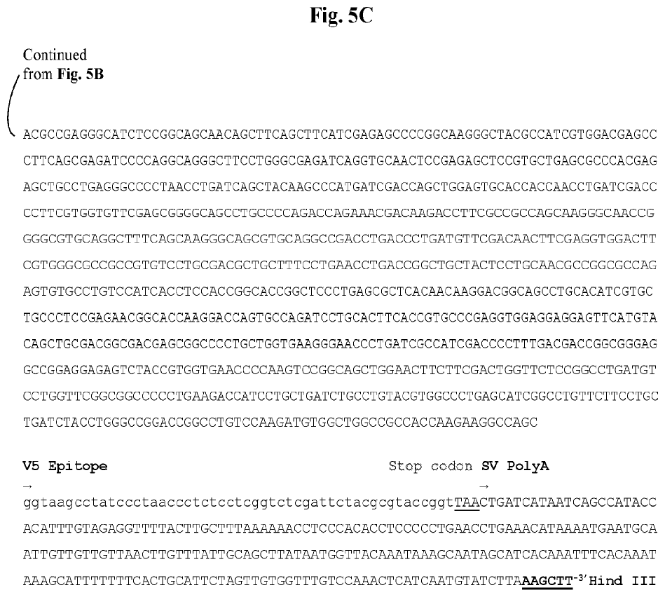

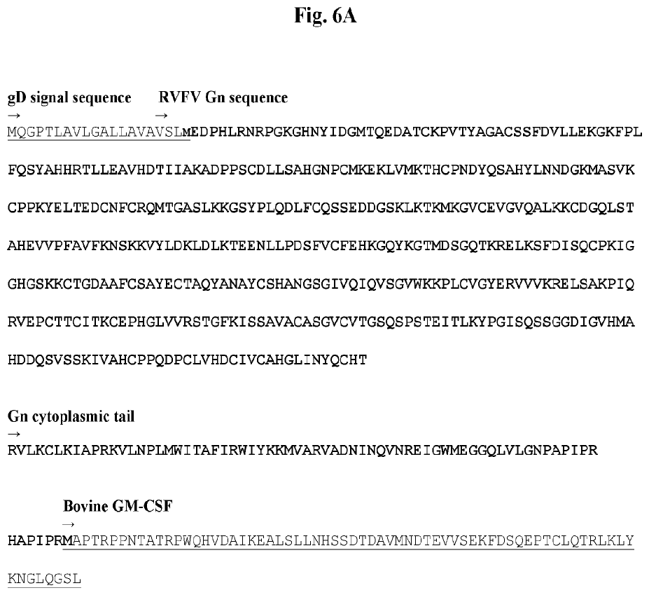

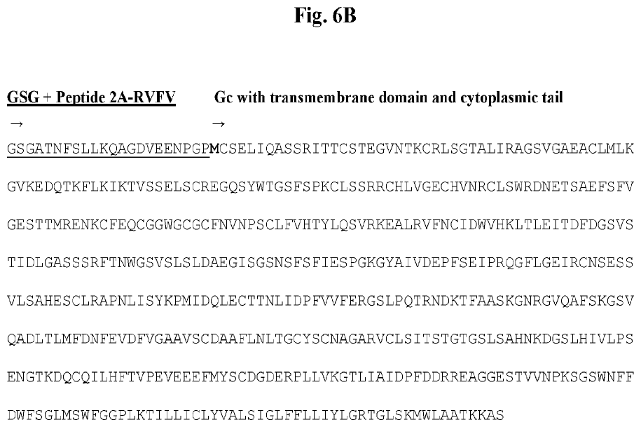

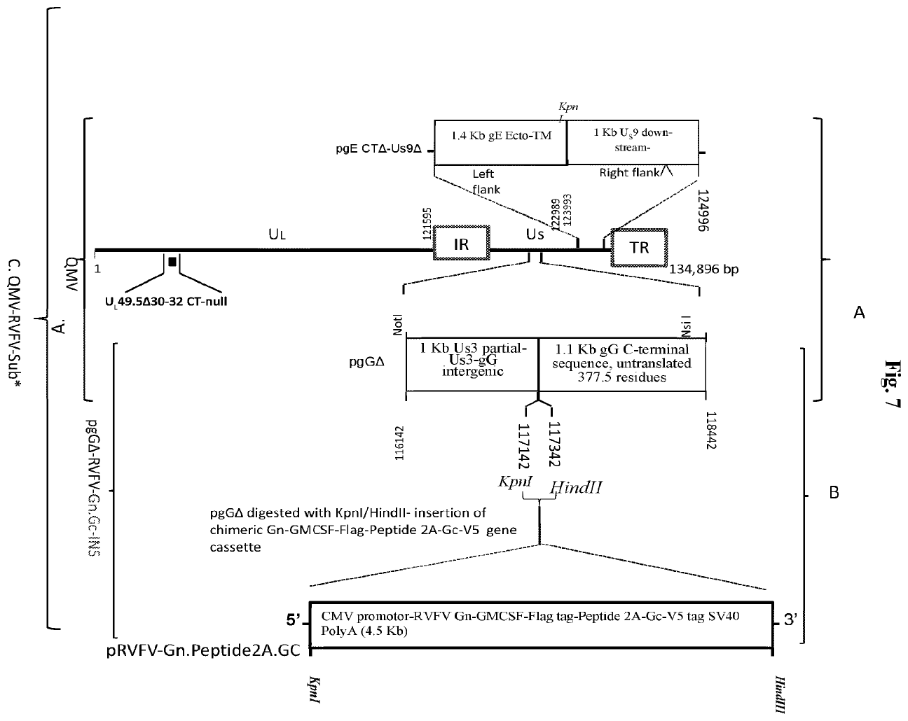

The strategy used to construct a BoHV-1 QMV expressing RVFV chimeric Gn-GMCSF-Peptide2A-Gc designated hereafter as BoHV-1 QMV-RVFV* is disclosed in the schematic FIG. 7. First, a 4.5 Kb chimeric gene sequence, designated as chimeric RVFV Gn-peptide2A-Gc gene, were synthesized commercially (FIG. 5A, 5B and 5C; Biomatic, Ontario, Canada). Chimeric RVFV Gn-peptide 2A-Gc is defined as SEQ ID NO: 10 and is designed to contain from 5′-3′ the following: a Kpn1 site, CMV IE promoter, Kozak sequence (SEQ ID NO: 4), predicted BoHV-1 gD signal peptide, RVFV Gn without the transmembrane domain but the ecto-domain (eGn), Gn cytoplasmic tail, Bovine GMCSF, Flag tag (SEQ ID NO: 6, variations of the Flag tag can also be used as long as it is recognized by commercially available anti-Flag antibodies), GSG sequence to improve cleavage efficiency, Peptide 2 A sequence, RVFV Gc region with transmembrane domain and cytoplasmic tail, V5 epitope, stop codon, SV polyA tail sequence and a HindIII site. The rationale for the chimeric Gn-GMCSF fusion protein is that the chimeric protein without the Gn transmembrane domain will be secreted. Additionally, the sequence encoding for the cytoplasmic tail of glycoprotein Gn was included with the ectodomain since it comprises motifs important for Golgi localization and processing. Similarly, the cytoplasmic tail and transmembrane domain of glycoprotein Gc also comprise important motifs, and as such were included in the chimeric gene construct. RVFV strain ZH-548 derived MP12 mutant vaccine virus genomic segment M codes for Gn (aa residues154-690) and Gc (aa 691-1206), respectively (GenBank accession #for ZH-548 segment M is DQ380206.1 and GenBank accession #for glycoproteins Gc and Gn is ABD38819.1). During RVFV replication, the two proteins are synthesized as a single protein; however, the protein is cleaved proteolytically by a cellular protease resulting in the Gn and Gc envelope proteins (Bouloy and Weber, 2010; Gerrard and Nichol, 2007). Therefore, the peptide 2A amino acid sequence (SEQ ID NO: 9: GSGATNFSLLKQAGDVEENPGP) was incorporated into the chimeric gene design, between the Gn-GMCSF-Flag and Gc-V5 sequences (FIGS. 5A-C and 6A-B), known to be a universal target for a cellular protease in all eukaryotic cells. FIG. 6A and 6B disclose the amino acid sequences of the chimeric polypeptide Gn-GMCSF as defined in SEQ ID NO: 11 and the chimeric polypeptide GSC+Peptide2A-Gc polypeptide as defined in SEQ ID NO: 12. The Gn polypeptide comprises the sequence of the Gn protein without the transmembrane domain but with the ectodomain and cytoplasmic tail which allow its secretion

Here, the nucleotide sequence of the chimeric RVFV envelope glycoproteins, Gn fused with GMCSF and Gc, has been codon optimized for cattle (FIG. 5A-C). The chimeric sequence can be codon optimized for it use in sheep-specific vaccine or other livestock. The rationale for separate sheep (Ovis aries) and cattle (Bos taurus) codon optimization of the RVFV Gn and Gc is that it will maximize their host-specific expression and therefore better immunogenicity.

### 2.10.2. Generation of the BoHV-1 QMV- RVFV* Mutant Virus

The 4.5 Kb KpnI-HindIII fragment synthesized above (FIGS. 5A-C) and defined as SEQ ID NO: 10 was cloned into the corresponding sites of the pgGA plasmid clone, resulting in pgGA-RVFV-Gn.Gc-INS (FIGS. 7A-B). The expression cassette encoding for the chimeric Gn-GMCSF-Gc protein (SEQ ID NO: 10) was cloned into the insertion site defined as SEQ ID NO: 3. To generate a recombinant BoHV-1 QMV- RVFV* mutant virus, linearized pgGΔ-RVFV-Gn. Gc-INS DNA (either with Notl or Nsil) was cotransfected with full-length BHV-1 QMV genomic DNA (FIG. 7C). Three putative recombinant viruses were plaque purified (3×) and verified by PCR and sequence analyses (Genelab, LSU).

### 2.11 Subcellular Localization of the Gn and Gc Recombinant Proteins

QMV-RVFV*-infected MDBK cells were fixed with a 3% solution of paraformaldehyde (PFA) for 20 min at room temperature (RT), permeabilized in a 0.2% Triton X 100 TBS solution for 15 min at RT. The cells were incubated n a blocking solution containing 3% BSA for 1 hour at RT. The primary antibodies were added in 1% BSA in TBS for a period of 2 hours at RT. The anti-VS monoclonal was used at a 1:200 dilution and the anti-Flag rabbit polyclonal was used at a 1:100 dilution. After incubation, the slides were washed. The secondary antibodies were then added 1% BSA in TBS for 1 hour at RT. The secondary antibody for the anti-VS Mab, the Alexa Fluor 488 donkey anti-mouse IgG, was added at a dilution of 1:400. The secondary antibody for the anti-Flag Mab, to the Alexa Fluor 647 donkey anti-rabbit IgG, was added at a 1:250 dilution. Nuclear staining was performed with DAPI at a 1:10,000 dilution in TBS for 10 min. All washes were done four times in TBS.

### 2.12. Animals and Experimental Design

Animal infection, handling, sample collection, and euthanasia protocols were previously approved by the LSU Institutional Animal Care and Use Committee. Fifteen, four to five-month-old cross-bred steer, bull, or heifer calves were obtained from a BVDV free supplier. Before inclusion in the study, the calves were tested for BoHV-1 and BVDV serum neutralizing (SN) antibody titers (4-<4) and nasal BVDV shedding to ensure BoHV-1/BVDV free status. Five calves with >4 BVDV-2 maternal SN antibody titers were selected for the control group. Five calves of the remaining 10 were allocated randomly to each of the two treatment groups. Group 1 (QMV-BVD* group) and group 2 (Bovi-Shield Gold® IBR-BVD; Zoetis; designated hereafter as the “Bovi” group) were housed in pens located in the School of Veterinary Medicine (closed) large animal isolation barn. Two pens, holding either two or three calves from each of the two vaccine groups, were well isolated (more than 100 feet apart). Foot baths were located at the main entry and in front of the entrance to each pen. Five calves selected for the control group or sham-vaccinated (group 3) had slightly higher maternal serum neutralizing titers (16-32). They were housed individually in separate pens at an open-air barn with a concrete floor and restricted access. The barn housing the control calves was approx. 100 yards away from the other barn, and a foot bath was located at the main entrance.

### 2.13. Vaccination and Challenge

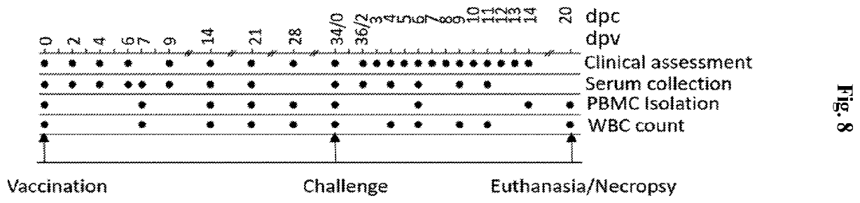

Vaccination, challenge, and sample collection scheme are shown in FIG. 8. After one week of acclimatization, QMV-BVD2* group was vaccinated both intranasally (IN) with 2×10⁷ PFU/nostril and subcutaneously (SQ) with 1×10⁷ PFU. The calves in the “Bovi” group were vaccinated SQ according to manufactures recommendations, and calves in the control group were sham vaccinated intranasally with 1.0 ml of cell culture media. The calves in QMV-BVD2* and “Bovi” groups received the Micotil® 300 (Tilmicosin; 20 mg/kg body weight) by SQ injection to prevent secondary bacterial infection. At 34 days post-vaccination (dpv), animals of all groups were challenged intranasally with a total of 2×10⁶ PFU (1×10⁶ PFU/ml/nostril) of ncp BVDV-2 890. The animals received Microtil® 300 subcutaneously as above. The experiment was terminated at 54 dpv/20 days post-challenge (dpc). Upon euthanasia, a complete necropsy was performed to investigate any gross pathological lesions and collect tissue samples.

### 2.14. Clinical Assessment of Calves

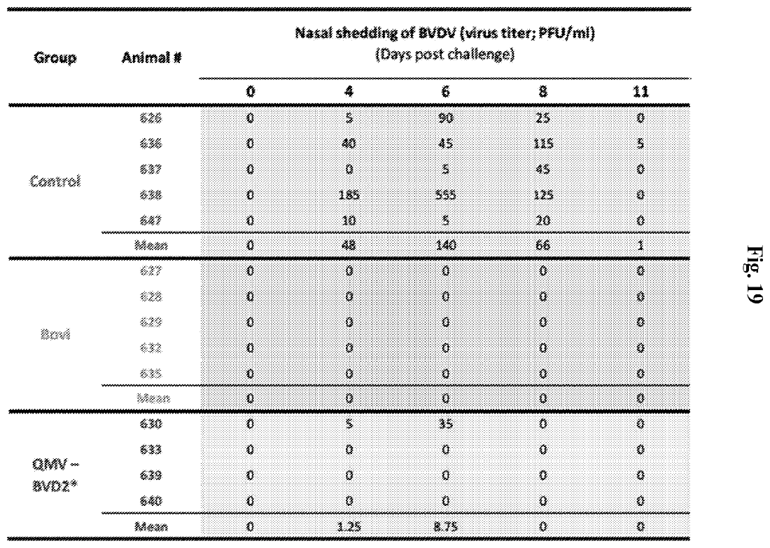

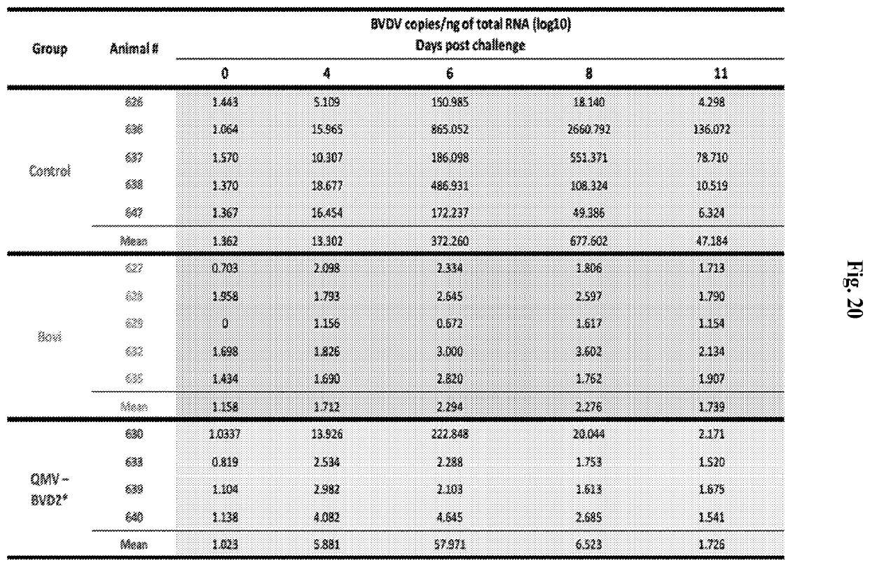

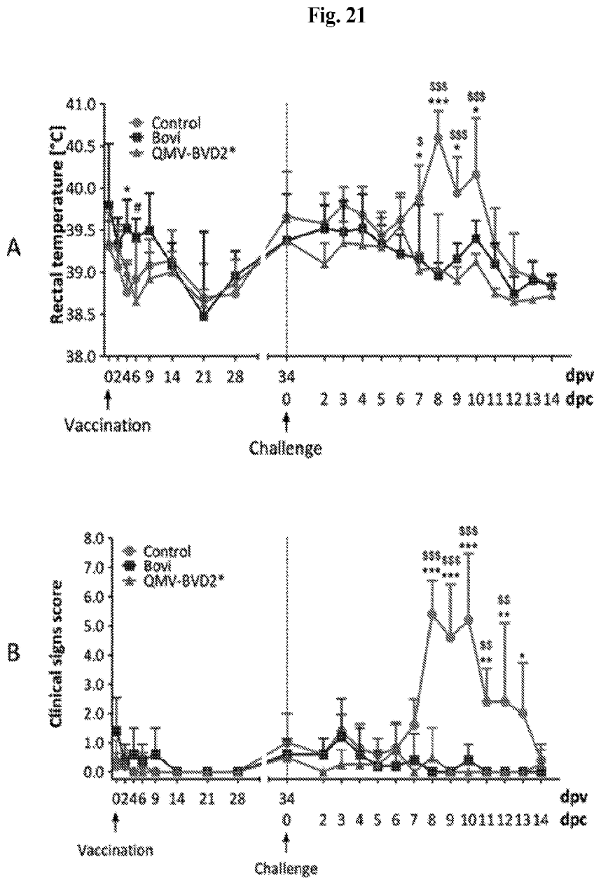

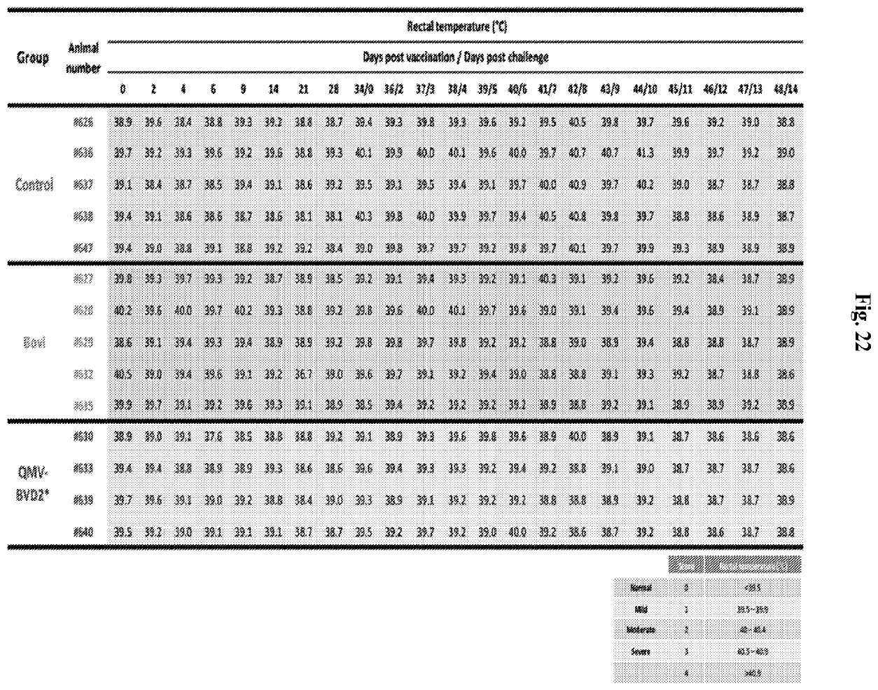

Calves were clinically assessed for the rectal temperature, feed, and water intake, on the day of vaccination (0 dpv) and 2, 4, 6, 9, 14, 21, and 28 dpv and on 34 dpv/0 dpc (FIGS. 21A and 22). Following the challenge with ncp BVDV-2 890, clinical signs were recorded daily until 14 dpc (FIGS. 21B and 24). The clinical assessment included rectal temperature, nasal and ocular discharge, dyspnea, coughing, lethargy, anorexia, mucosal and oral lesions, and diarrhea. Clinical scores for each animal were calculated based on the criteria listed in FIG. 23.

### 2.15. Sample Collection and Processing

The schedule of EDTA-blood, serum, and nasal swab collection is shown in FIG. 8. Nasopharyngeal swabs were collected in 1 ml of cell culture media supplemented with 2× antibiotic/antimycotic solution, on 0, 2, 4, 6, 7, 9, 14, and 21 dpv, and 0, 2, 4, 6, 8, and 11 dpc (FIG. 8). The samples were processed and stored at −80° C. Virus titrations by plaque assay were performed as described above in 2.4.

### 2.16. Isolation and Freezing of PBMC

PBMCs were isolated using Ficoll-Paque (Ficoll-Paque·8 PLUS, GE health, NJ, USA) density-gradient centrifugation as previously described [37]. For freezing, isolated PBMCs were resuspended in 10% FBS—RPMI-1640 medium containing 10% dimethyl sulfoxide (DMSO; Sigma-Aldrich) at a concentration of 5×10⁶ cells/ml. Aliquots of PBMCs were subjected to slow freezing at −80° C. (overnight) before transferring to a liquid nitrogen tank for long-term storage.

### 2.17. Leukocyte Counting in Whole Blood-EDTA Samples

For counting the leukocytes in whole blood-EDTA samples, an automatic hematological analyzer (Advia 120: Siemens, Tarrytown, NY) was used. On the day of challenge (0 dpc) and on 4, 6, 8, 11, and 14 dpc, total leukocyte counts were determined and recorded. In addition, the percent decline in leukocyte numbers in each calf was calculated as follows and described earlier [38]:

$$\% decline of Leukocyte count of an animal “ X ” = 100 - \frac{Lowest leukocyte count of an animal “ X ”}{Leukocyte count of the animal on 0 dpc} \times 100$$

A decline in the leukocyte count of more than 25% was considered leukopenia (Beer et al., 2000).

### 2.18. Virus-Neutralization Assay

Sera were heat-inactivated at 56° C. for 30 min. 250 ill of BoHV-1 wt Cooper or BVDV-2 125 virus suspension containing approx.100 PFUs/100 μl were preincubated with 250 μl of serial four-fold serum dilutions (for BVDV-2) or serial two-fold serum dilutions (for BoHV-1) at 37° C. for 2 h. Similarly, 250 μl of plain cell culture media was incubated with 250μ1 of the respective virus suspensions in 6-8 tubes (virus control) and incubated at 37° C. for 2h. 200 Two hundred microliters of the serum-virus mixture from each serum dilution were added to two wells (duplicate) of 24-well cell culture plates containing confluent MDBK cells. For the virus control, 200 μl of virus-media mixtures were added to 6-8 wells of 24-well plates. After 2 h incubation at 37° C., 0.8 ml of 1.6% CMC in DMEM was added to each well. The plates were incubated in a CO₂ incubator at 37° C. for two days for the BoHV-1 and 4 days for the BVDV-2 plaque assays. After fixing the cells with 10% formalin (1-2 h) and washing with tap water, the cells were stained with 0.35% crystal violet solution (20 min). The viral plaques in the serum-virus mixture wells and their respective virus control wells were counted under a microscope. The reciprocal of the highest dilution of each serum that inhibited/neutralized 50% of the average number of the respective control virus plaques, but not less than 40-45 plaques, was reported as the virus-neutralization titer.

### 2.19. Reverse Transcription Quantitative Real-Time PCR (RT-qPCR) for BVDV Viremia

To detect BVDV, RT-qPCR on post-challenge PBMCs samples was performed. Briefly, total RNAs were extracted from the PBMCs of calves at 0, 4,6,8, and 11 dpc, using RNA easy extraction kit (Qiagen) according to the manufacturer's recommendations. cDNA was generated from 250 ng of total RNA followed by RT-qPCR using the VetMax-Gold BVDV detection kit (ThermoFisher, MA, USA, U.S. Pat. No. 4,413,938). RNA isolation was performed two times, and the RT-qPCR analysis was repeated three times in duplicate for each sample. BVDV genome load was calculated following the manufacturer's instruction. According to the manufacturer's instruction, 1 μl of the positive control (25×BVDV RNA) contains 10,000 copies of BVDV. To generate a standard curve, 8 μl of the positive control were serial diluted 10 fold. Standard samples corresponding to 4, 40, 400, and 4000 copies were included in each PCR analysis. BVDV copy numbers in each sample were calculated according to the standard curve's CT-values, divided by 250 to BVDV genome in one ng total RNA. All samples that had a copy number lower than the highest copy number detected in samples from 0 dpc (Threshold of 2.32 copies/ng total RNA) were evaluated as negative.

### 2.20. BVDV-Specific Cellular IFN-γ and Proliferation Responses

At day 0 and 14 post-vaccination and day 6 post-challenge, IFN-γ responses in PBMCs were evaluated by enzyme-linked immunospot (ELISPOT) assay. The assay was performed using Bovine IFN-γ ELISpot BASIC (ALP) kit (Mabtech, Stockholm, Sweden, #3119-2A) as per the manufacturer's instruction and as described previously [33, 39]. Briefly, 0.25×10⁶ whole-blood-derived PBMCs were seeded in triplicate wells of MultiScreen-IP plates (MilliporeSigma™, #MAIPS4510) with whole heat-killed BVDV virus [CA0401186a (CA), TGAC, A125 or 1373] in a final volume of 100 μl complete RPMI 1640 medium. The positive control was 2.5 μg/ml concanavalin A (ConA), whereas medium alone was used as a negative control. The spots were quantified with an ELISPOT reader, Cellular Technology Limited (CTL, OH, USA) ImmunoSpot® S6 Analyzer. The results were presented as the mean number of BVDV-specific IFN-γ⁺ spot-forming cells (SFC) per 10⁶ PBMCs after deducted background medium counts.

BVDV-specific PBMC proliferation responses on 14 dpv and 4 dpc were determined using ³H-Thymidine incorporation assay as previously described [33, 39]. Briefly, 0.5×10⁶ whole-blood-derived PBMCs were cultured for 72 h at 37° C. in triplicate wells of round-bottom 96-well plates in a total volume of 100 μl of complete RPMI 1640 medium containing 10 μg/ml of whole heat-killed BVDV virus. The positive control was 1.25 μg/ml ConA, whereas medium alone was used as a negative control. Cells were labeled with 0.25 μCi of ³H-thymidine for 12 h and then harvested using a semi-automatic cell harvester (Perkin Elmer, MA, USA), and the incorporated ³H-thymidine was counted with a Micro-Beta liquid scintillation counter (Perkin Elmer). The incorporation of ³H-thymidine by the proliferating PBMCs was presented as mean counts per minute (CPM) of triplicate wells after deducting the background medium counts.

### 2.21 Euthanasia, Necropsy and Pathology

Calves were euthanatized with xylazine and pentabarbitol 20 dpc (FIG. 8). Complete necropsies were performed. Samples were collected from the lungs, systematically from cranial and caudal lobes with additional sections from gross lesions. Lungs were photographed, and lesions were recorded. Samples were also collected from the kidney, liver, spleen, mesenteric lymph node, tonsil, bronchial lymph node, cervical lymph node, and bone marrow. Samples were fixed in 10% buffered-neutral formalin. Hematoxylin-eosin (HE) stained slides were obtained from paraffin-embedded tissues by routine methods.

All tissues were evaluated and scored by a single veterinary pathologist, who was blinded to treatment. Tissues, except lungs, were scored on a scale of 0-4 (0=normal, 1=minimal, 2=mild, 3=moderate, and 4=severe) on multiple parameters. Parameters included acute inflammation, chronic inflammation, and necrosis for all tissues with additional tissue-specific parameters, such as glomerular changes for kidneys, lymphoid depletion, and hyperplasia for all lymphoid organs, sinus histiocytosis for lymph nodes, and myeloid/erythroid hyperplasia in the bone marrow. All lung sections were individually scored on a 0-3 scale (0=normal, 1=mild, 2=moderate, and 3=severe). Bonchi/bronchioles, parenchyma, and septa/pleura were evaluated in each section.

### 2.22 Statistical Analysis

### 2.22.1 Nasal Virus Shedding and Viremia

All data were expressed as means±standard deviation. Statistical analyses were performed using GraphPad PRISM® software version 5.04. The two-way analysis of variance (ANOVA) followed by Bonferroni posts-tests to compare replicate means by row were performed. A value of p<0.05 was considered statistically significant.

### 2.22.2 Calculation of Outliers

Outliers in data point that differs significantly from other observations were estimated by Grubb's test (generalized extreme studentized deviate method) with alpha level of 0.05 using GraphPad PRISM® software.

### 2.22.3 Cellular Immune Response

Nonparametric Kruskal-Wallis test with Dunn's multiple comparisons test was used to analyze the significant differences between groups. Post-vaccination and post-challenge, the significance of the differences in BVDV-specific immune responses (cellular IFN-γ and proliferation responses) were compared among all groups. Statistical analysis was performed using GraphPad Prism 7 (Version 7.04, GraphPad Software, Inc. CA, USA). A significance level of P<0.05 was used for all analyses.

### 2.22.4 Histopathology of Lung Sections

A nonparametric rank test of factorial ANOVA with repeated measures was performed to detect the differences in the three vaccinated groups' efficacy levels, adjusted by Aligned Rank Transformation [23]. The rationale of this approach is to allow nonparametric factorial analyses when handling repeated measures [40]. This approach is more robust to test sophisticated data structure than other traditional nonparametric tests [41]. The adjustment method ART relies on alignment and ranking step before using F-tests. Therefore, ART is similar to the parametric ANOVA, except that the response variable may be continuous or ordinal and is not required to be normally distributed. Post-hoc pairwise comparisons were conducted and the alpha levels were adjusted by Tukey method.

### 3.0 Results

### 3.1. Characterization QMV-BVD-2* Recombinant Vector

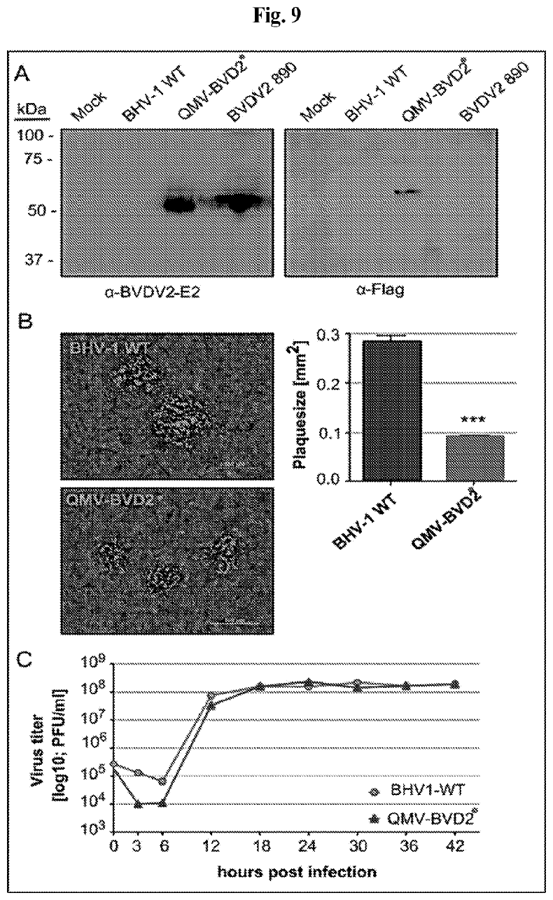

FIG. 9 shows characterization of the QMV-BVD-2* recombinant virus in which A shows an Immunoblot analysis of QMV-BVD2* expressing chimeric BVDV2 E2 and Erns-GMCSF-Flag fusion proteins by using anti BVDV2 E2-specific (left panel) and anti-Flag-specific (right panel) antibodies, respectively; B. shows plaque size analysis of QMV-BVD2* compared to that of BoHV-1 wt. Shown are the pictures of areas containing two-three representative plaques of each virus. The bar graph shows the average plaque size of at least 50 plaques with SD (***P<0.001); and C. graphically shows the one-step growth analysis of QMV- BVD2* compared with the BoHV-1 wt.

### 3.1.1. QMV-BVD2* Virus Expresses the Chimeric BVDV-2 E2 and Erns-GMCSF-Flag Proteins

Sequence analysis of the Erns-GMCSF and E2 chimeric genes and their flanking, QMV-BVD2* sequence (approx. 1000 bp on each side) validated the chimeric gene sequence's integrity and their insertion at the gG and gECT-Us9 deletion loci, respectively (data not shown). Further, the expression of BVDV-2 E2 antigen and chimeric Erns-GMCSF in QMV-BVD2*-infected cell lysates was verified by SDS-PAGE/Western immunoblotting. For this, QMV-BVD2*, BoHV-1 wt-and BVDV-2 890-infected cell lysates were tested for chimeric BVDV-2 E2 and Flag-tagged Erns- GMCSF expression. Western Blot analysis using BVDV E2-specific mAb recognized an approx. 53-55k D bands both in QMV-BVD2* and BVDV-2 890-infected cell lysates (FIG. 9A, left panel). In contrast, the anti-Flag mAb recognized a 56 kD band in the QMV-BVD2*-infected cell lysate (FIG. 9A, right panel). As expected, the E2 mAb did not bind to any band in the BoHV-1 wt-infected cell lysate, and the anti-Flag mAb did not recognize any protein band in the BoHV-1 wt and 890-infected cell lysate (FIG. 9A). As noted earlier, the E2 chimeric protein is fused to V5 epitope and 6×His tags at the carboxy end. The combined molecular mass of V5 and 6×His is approx. 2.2 kD, which was not large enough to make a noticeable shift of the chimeric E2 band of QMV-BVD2* compared to the authentic E2 band of the BVDV-2 strain 890 (FIG. 9A). The estimated molecular mass of chimeric Erns (25.7 kD)-GM CSF (17.13kD)-Flag (1.02 kD) protein is 43.8 kD (https://web.expasv.org/cgi-bin/compute_pi/pi_tool). However, as noted above, the anti-Flag mAb detected an approx. 56 kD band on the Western blot containing the QMV-BVD2*-infected cell lysate (FIG. 9A). The increase in the chimeric Erns-GMCSF-Flag protein's size is most likely due to glycosylation because the amino acid sequence analysis (NetNGlyc 1.0 server, Technical University of Denmark; http://www.cbs.dtu.dk/cgi-bin/webface2.fcgi!jobid) predicted at least six major, potential N glycosylation sites (data not shown).

### 3.1.2. QMV-BVD2* Virus Produces Smaller Plaques but Replicates with Similar Kinetics and Yield Compared with the BoHV-1 wt

The QMV-BVD2* virus produced smaller plaques than BoHV-1 wt (FIG. 9B), and the plaques were very similar to those of the BoHV-1 TMV virus described earlier. The bar graph shows the average plaque size of at least 50 plaques with SD (***P<0.001). (FIG. 9B, right panel) The one-step growth curve results showed that the growth kinetics and virus yield of QMV-BVD2* are similar to that of BoHV-1 wt (FIG. 9C).

### 3.2. Characterization of QMV-RVFV* in Infected Cells

It was important to engineer a BoHV-1 QMV vector virus expressing the chimeric Gn fused with GM-CSF and Gc proteins. The rationale for the chimeric Gn-GMCSF fusion protein is that the chimeric protein without the Gn transmembrane domain but with the ectodomain and cytoplasmic tail will be and properly localized and processed in the cell and subsequently secreted. The objective was that RVFV protective antigens expressed by the BoHV-1 QMV will be processed similarly as in RVFV-infected cells without affecting BHV-1 QMV replication.

### 3.2.1 Expression of the Cleaved Gn and Gc Recombinant Proteins

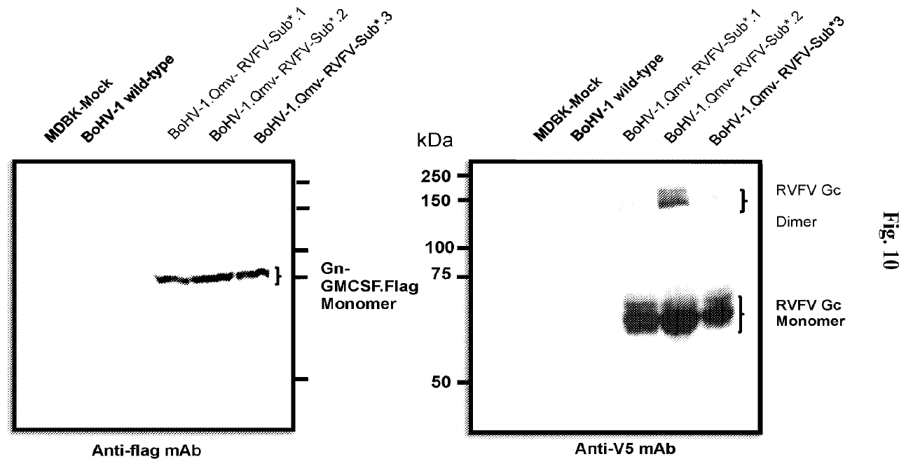

It was important to verify that peptide 2A cleavage of the chimeric Gn-Gc protein expressed by the putative recombinant viruses worked as designed, SDS-PAGE/Western immunoblotting analysis of infected cell lysates were performed as disclosed in 2.8. The results in FIG. 10 show that all three putative recombinants generated (as described in 2.10) expressed the proteolytically cleaved chimeric Gn-Flag and Gc-V5 proteins, namely QMV-RVFV-1, 2 and 3 (FIG. 10). As expected, the anti-Flag antibody detects a 70 kDa Gn-GMCSF-Flag monomer (FIG. 10, left panel) while the anti-VS antibody detects the 56 kDa (monomer) and 112 kDa (dimer) of Gc-V5 proteins (FIG. 10, right panel). These results demonstrated that the chimeric Gn-P2A-Gc protein was cleaved specifically at the peptide 2A site by a cellular protease resulting two individual proteins. For comparison mock and BoHV-1 wild-type-infected MDBK cell lysates are included (FIG. 10).

### 3.2.2 Subcellular Localization of the Gn and Gc Recombinant Proteins

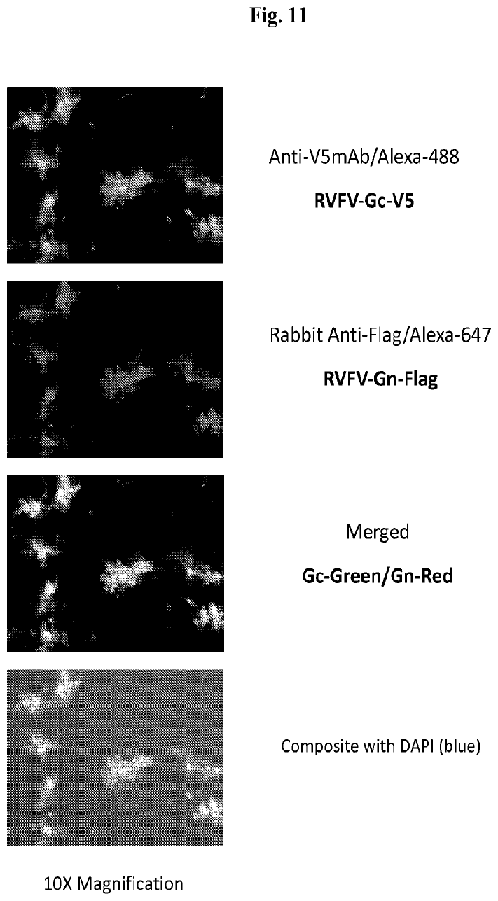

From the immunoblot experiment described above, one putative recombinant QMV-RVFV*.2 was selected for further analysis of subcellular localization of the Gn-GMCSF-Flag and Gc-V5. The results of immunofluorescence assays show that the Gn-GMCSF-Flag (B/C) and Gc-V5 (A/C) distributions are predominantly in the perinuclear region but also diffuse within the cytoplasm (FIG. 11). Notably, they colocalized extensively in the perinuclear area of the infected cells as visible in panel D of FIG. 11. Based on these results, we conclude that the QMV-RVFV* virus expresses both the chimeric Gn-GMCSF-Flag and Gc-V5 as designed.

### 3.3 Pathogenicity and Nasal Virus Shedding Following IN/SQ Vaccination with Live QMV-BVD2* Subunit Vaccine

Following vaccination with QMV-BVD2* (IN/SQ) and “Bovi” (SQ), the calves remained clinically normal regardless of the vaccine used. As expected, 2 dpv, all the QMV-BVD2* vaccinated animals (5/5) shed the vaccine virus with an (average titer 2.26×10² PFU/ml) (data not shown). On the 4 dpv, four animals (4/5) shed the virus (average titer 2.7×10³ PFU/ml) (data not shown). On 6, 7, and 9 dpv QMV-BVD2* vaccine virus could not be isolated from any of the QMV-BVD2* vaccinated animals. None of the “Bovi” vaccinees and the negative control calves shed any of the vaccine viruses in the nose. One calf (#648) in the QMV-BVD2* group developed diarrhea and fever on 28 dpv due to an unknown cause. The calf was treated with antibiotics and physiological saline infusion. The calf was later euthanized prior to the challenge.

### 3.4 Post-Vaccination BoHV-1 Serum Neutralizing Antibody Titers in the QMV-BVD2* Group was Slightly Lower than that of the “Bovi” Group, but BVDV-2 Neutralizing Antibody Titers in the “Bovi” Group was Considerably Better than in QMV-BVD2*

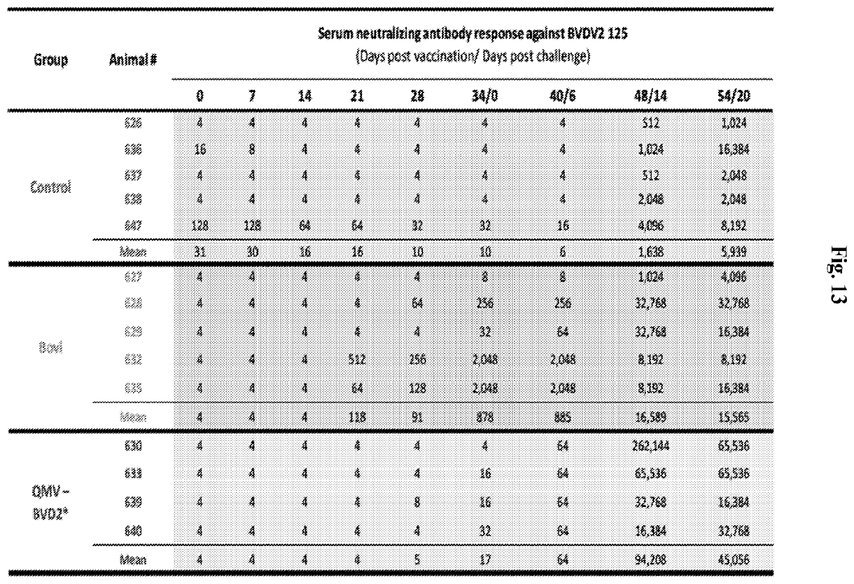

On the day of vaccination (0 dpv), mean BoHV-1- and BVDV-2-specific maternal antibody titers in both QMV-BVD2* and “Bovi” vaccine groups were approx. 4 (FIG. 12). However, the control calves had a relatively high titer (approx. 31) against BVDV-2 (FIG. 12, bottom panel and FIG. 13) but not against BoHV-1 (FIG. 12, top panel). Following vaccination, the average BoHV-1-specific neutralizing titers in the QMV-BVD2*-vaccinated group rose to 9 (two-fold) by 34 dpv (0 dpc or on the day of the challenge) (FIG. 12, top panel). In the case of “Bovi” treatment group, the corresponding increase in the neutralizing titers was slightly more than three-fold (from 4 to 14) (FIG. 12, top panel).

As depicted in FIG. 12, bottom panel and FIG. 13, the mean BVDV-2-specific maternal neutralizing antibody titers in the control animals declined to 10 by 34 dpv (0 dpc). In contrast, by 34 dpv, the BVDV2-specific neutralizing titers increased four-fold, from 4 to 17 (seroconverted) in QMV-BVD2* group and the corresponding titers in the “Bovi” group rose 210-fold (from 4-878) (FIG. 12, bottom panel and FIG. 13). SN antibody titers were determined by standard plaque reduction assay. The top panel shows BoHV-1-specific SN antibody titer against BoHV-1 Cooper (Colorado-1) strain following vaccination. The data represent the mean plus standard deviation. Significant differences in BVDV-2 neutralizing antibody titer were seen between the different groups. Two-way ANOVA followed by Bonferroni post-tests was used to compare replicate means by row with the symbol ($$$) showing a significance p<0.001 between Control and QMV-BVD2* group. The symbol (#) shows a significance p<0.05, and the symbol (###) shows a significance p<0.001 between Bovi and QMV-BVD2* groups.

### 3.5 QMV-BVD2* Vaccinated Calves Induced Higher BVDV Cross-Reactive (Types 1 and 2) IFN-γ Responses than that of the “Bovi” Group

FIG. 14 shows Pre-vaccination, Post-vaccination, and post-challenge BVDV-1 (A) and BVD2 (B) strain-specific IFN-γ cellular responses. IFN-γ secreting PBMC responses against BVDV-1 (CA and TAGC) and BVDV-2 (A125 and 1373) strains were determined by IFN-γ ELISPOT assays. The response is presented as IFN-γ⁺ SFC/10⁶ PBMCs. Medium alone served as the negative control, and the data shown are normalized by deducting media background counts.

IFN-γ responses in the PBMCs collected on 0 and 14 dpv against heat-killed BVDV-1 strains CA (ncp) and TGAC (cp), and BVDV-2 strains 1373 (ncp) and A125 (cp), and ncp1373) strains by ELISPOT assays were determined (Fig. FIG. 14A and B). All the animals were negative on 0 dpv; however, on 14 dpv, BVDV-1- and -2-specific IFN-γ responses were detected in the calves vaccinated with either QMV-BVD2* or “Bovi”, but not in the negative controls. Medium alone served as the negative control, and the data shown in both panels are normalized by deducting media background counts. The group mean is represented by bar and the asterisks denote statistically significant differences between the groups (*P<0.05) (FIG. 14A and B).

Notably, on 14 dpv, QMV-BVD2*-vaccinated calves had the highest mean IFN-γ responses against both the BVDV-1 CA (88) and TGAC (144*) strains (FIG. 14A). Notably, the mean TGAC-specific IFN-γ response detected in the QMV-BVD2*-vaccinated calves (144*) were significantly higher (*P<0.05) than the mean response seen in the negative control calves (FIG. 14A). Similarly, post-vaccination BVDV-2 (125 and 1373)-specific IFN-γ responses in QMV-BVD2*-vaccinated calves were the highest among the three treatment groups (FIG. 14B). In addition, mean responses detected in “Bovi” and QMV-BVD2*-vaccinated calves for the two BVDV-2 strains (represented by a bar) were significantly higher (*P<0.05) than the mean responses in the negative control calves (FIG. 14B).

### 3.6 QMV-BVD2* Vaccinated Calves Induced Higher Cross-Reactive (BVDV-1 and -2) Recall Cell (PBMC) Proliferation Responses than that of the “Bovi” Group

FIG. 15 shows Post-vaccination and post-challenge BVDV-1 (A) and BVDV-2 (B) strain-specific proliferation of the PBMCs. PBMC responses against BVDV-1 and BVDV 2 strains were determined by cell proliferation assays. The incorporation of 3H-thymidine by the proliferating PBMCs is presented as CPM. Medium alone served as the negative control, and the data shown is minus media background counts.

Recall cell proliferation responses against BVDV-1 and -2 were evaluated on day 14 post-vaccination (FIG. 15A and B). Among the three treatment groups, the highest mean BVDV-1-specific cell proliferation response was detected in QMV-BVD2* vaccinees (FIG. 15A). However, no significant difference was detected between the mean responses of the QMV-BVD2* vaccinees against two BVDV-1 strains compared with the mean response in the control group (FIG. 15A). Nevertheless, the mean proliferation response of the QMV-BVD2* vaccines was two-fold higher than the “Bovi” vaccinees against the TGAC strain (FIG. 15A). The group mean is represented by bar and the asterisks denote statistically significant differences between the groups (*P<0.05 and ** P<0.01).

The QMV-BVD2* experimental vaccine also generated the highest mean BVDV-2 specific cell proliferation responses on 14 dpv against A125 and 1373 strains among the three treatment groups (FIG. 15B). Although the QMV-BVD2* responses compared with that of the “Bovi” group were 2-fold higher, the proliferation responses were not significant relative to the control group (FIG. 15B).

### 3.7 QMV-BVD2* Vaccinated Calves Induced Higher Memory Serum Neutralization Antibody Response After Challenge with BVDV-2 than that of the “Bovi” Group

Following challenge with ncp BVDV-2 890, average neutralizing antibody titer in control calves decreased from 10 on 0 dpc to 6 on 6 dpc, which was most likely the decline in maternal antibody titer (FIG. 13). In the commercial “Bovi” vaccine group, the average neutralizing titer against BVDV-2 did not rise after the challenge at 6dpc (increased from 878 at 0 dpc to 885 on 6 dpc) or did not seroconvert (FIG. 12, bottom panel and FIG. 13). In contrast, in the QMV-BVD2* vaccinated group, the BVDV-2-specific average neutralizing titer increased 17 at 0 dpc to 64 at 6 dpc, a 3.8-fold (fourfold) increase or seroconverted (FIG. 12, bottom panel and FIG. 13). Remarkably, at 14 dpc, the average neutralizing titer in the QMV-BVD2* group was 94,208, a 5,500-fold increase compared to 0 dpc (FIG. 12, bottom panel and FIG. 13). In contrast, the average neutralizing titer of the “Bovi” and sham (control) vaccinated groups, on 14 dpc, were 16,589 and 1,638, which were six- and 58-folds less than that of the QMV-BVD2*, respectively (FIG. 12, bottom panel and FIG. 13). Therefore, upon BVDV-2 challenge, the relative recall serum neutralizing antibody responses of the QMV-BVD2* vaccinated calves at 14 dpc was more robust than that of the commercial “Bovi” vaccinated calves.

### 3.8 QMV-BVD2* Vaccinated Animals had the Highest BVDV Cross-Reactive Post-Challenge Cellular IFN-γ and Proliferation Responses

At 6 days post-challenge PBMCs from the QMV-BVD2* vaccinated animals had the highest mean BVDV-1- and BVDV-2-specific recall IFN-γ responses amongst the three treatment groups (FIG. 14). However, the differences among the groups were not significant. (FIG. 14A and B). Overall, the mean IFN-γ responses recalled post-challenge in the QMV-BVD2*-vaccinated animals were at least two-fold higher than that of the “Bovi” vaccinees against BVDV-1 and -2 strains (FIGS. 14A and B).

Notably, upon BVDV-2 challenge, QMV-BVD2* vaccine generated the highest mean cross-reactive (BVDV-1- and -2) recall cell proliferation responses among the three treatment groups (FIGS. 15A and B). Specifically, in comparison to the negative controls, the QMV-BVD2* vaccinees had significantly higher post-challenge recall cell proliferation against both BVDV-1 strains: CA (*P<0.05) and TGAC (**P<0.01) (FIG. 15A) and against BVDV-2 strain 125 (**P<0.01) (FIG. 15B). Although the proliferation responses recalled in the QMV-BVD2*-vaccinees against BVDV-2 strain 1373 was the highest, the mean proliferation level compared with that of the control calves was not significant (FIG. 15B). Nevertheless, the mean cellular proliferation response in the QMV-BVD2* treatment group was at least two-fold higher than that of the “Bovi” treatment group against the 1373 strain (FIG. 15B).

### 3.9 QMV-BVD2* Vaccinated Calves had a Mild and Brief Period of Leukopenia after the BVDV-2 Challenge

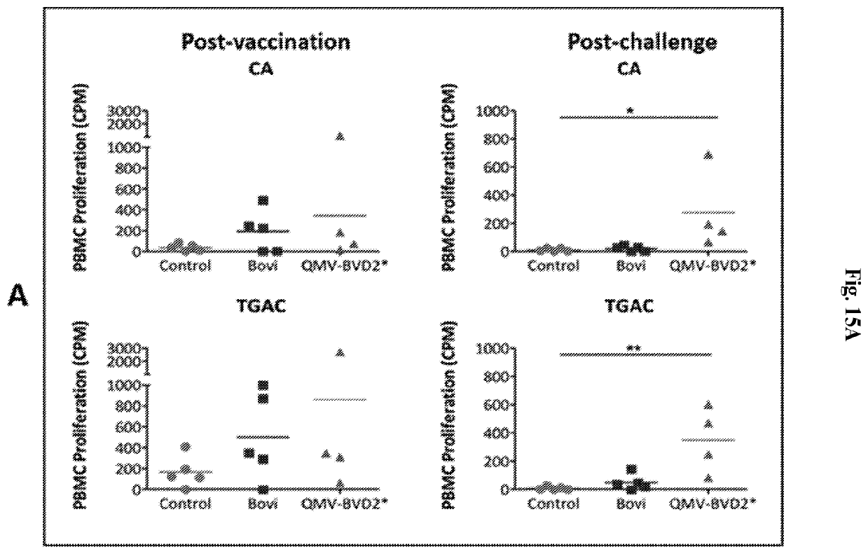

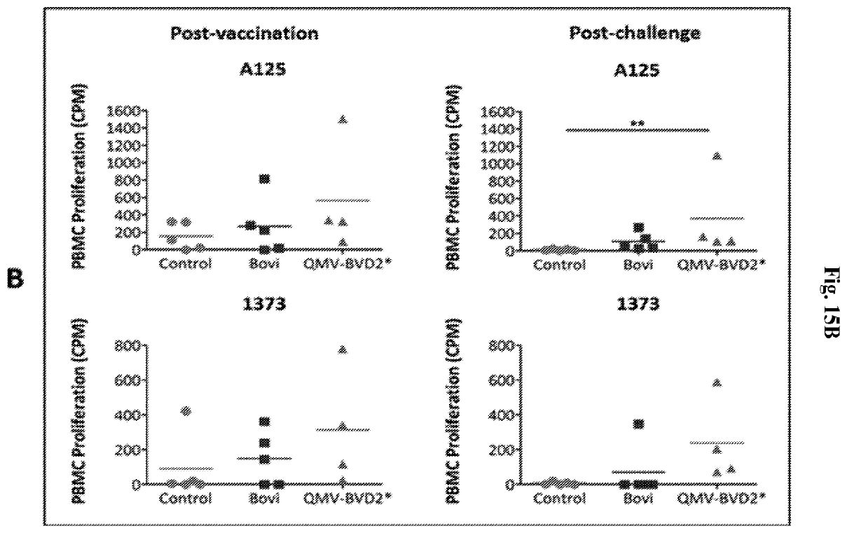

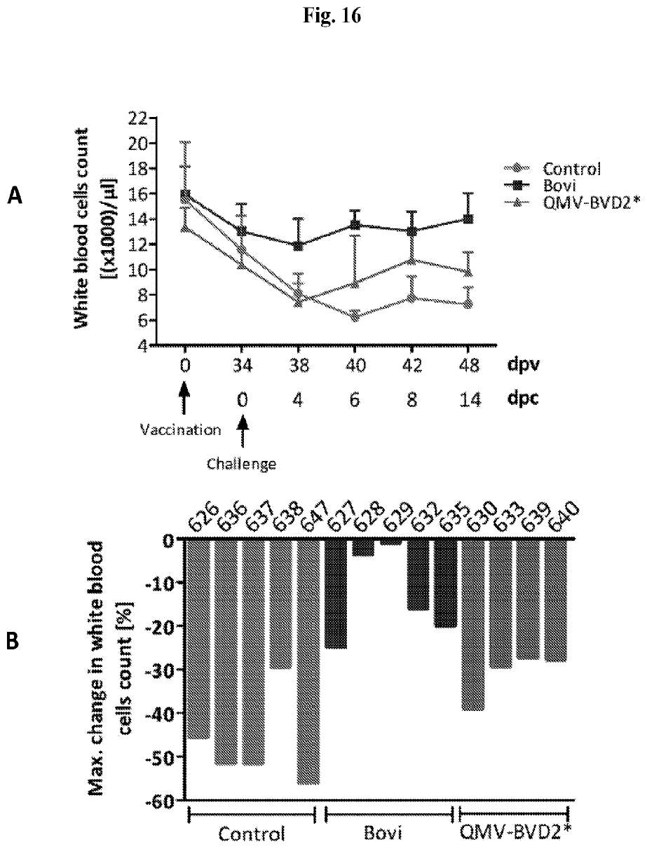

On the day of challenge (0 dpc) and on 4, 6, 8, 11, and 14 dpc, total leukocyte counts were determined and recorded (FIG. 16A). Based on the criteria used to calculate the percent decline in leukocyte numbers in material and methods, all control animals had leukopenia from 4 dpc until 14 dpc. Notably, the highest percentage decline in the control animals ranged between 29-56%, with a 47% mean percentage decline in the group (FIG. 16B). Based on the percent decline in leukocyte count, three calves from the QMV-BVD2* treatment group had mild leukopenia (approx. 30% decline, which is 5% more than the threshold) while calf #630 had a drop of 13%, which lasted for three days, 4 dpc-6 dpc (FIG. 16B). None of the “Bovi” vaccinated calves had leukopenia (FIG. 16B). In the QMV-BVD2* treatment group, there was a brief drop in leukocyte counts on 6 dpc from 10.2×10³ to 8×10³/μl (FIG. 16A), which was still within the average values. However, the mean leukocyte counts rose back to 9.5×10³/μl by 6 dpc in three of four calves (FIG. 16A). Notably, calf #630 had a low leukocyte count of 6.3×10³/μl on 0 dpc (FIG. 17), which is at the lower end of a normal range (6-12×10³) and 2×10³ fewer than the mean, standard leukocyte count of 8×10³. This calf had leukopenia both on 4 dpc and 6 dpc, but the number rose back to 10×10³/μl on 8 dpc (FIG. 17).

### 3.10 Except for one Outlier in the QMV-BVD2* Group, Both QMV-BVD2* and “Bovi” Vaccinated Groups Did Not Shed the Challenge Virus in the Nasal Secretion

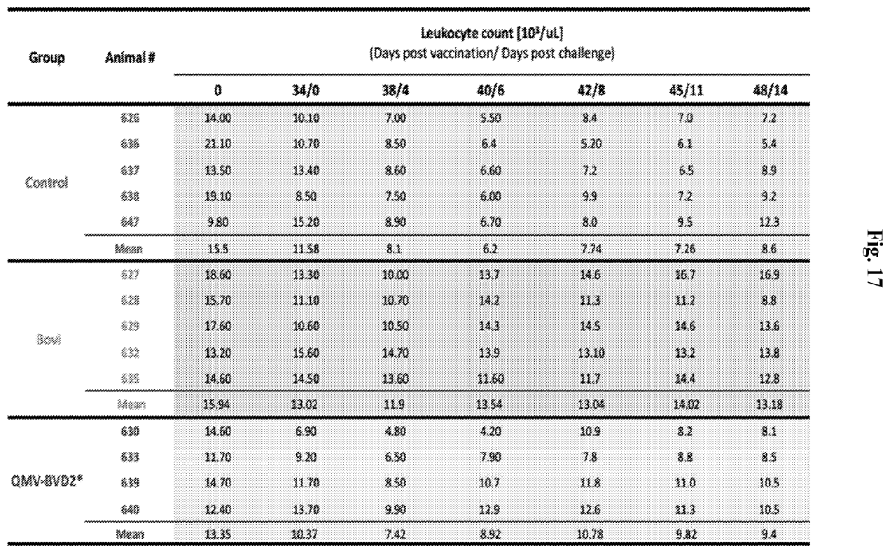

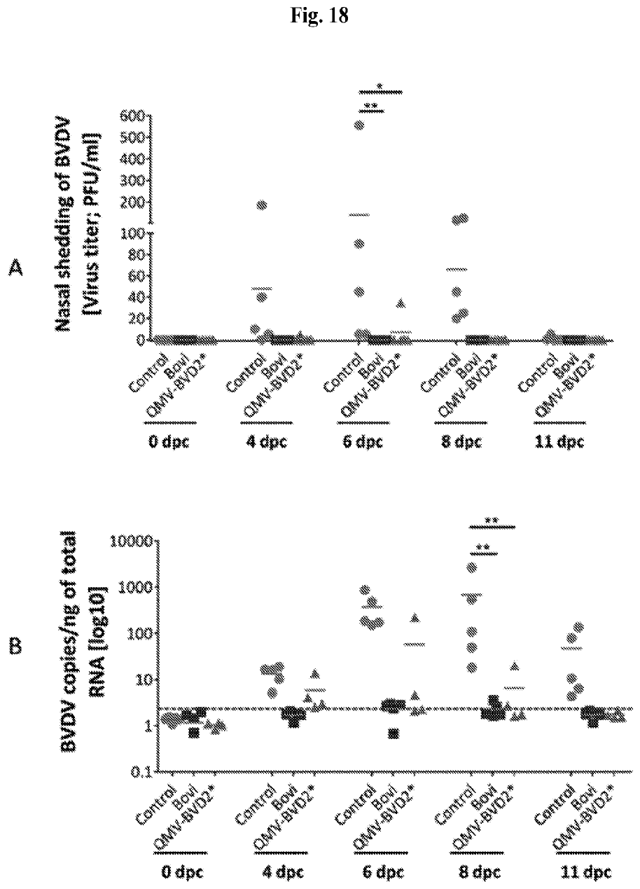

On 4 dpc, three control animals shed low (5-40 PFU's/ml) to moderate amounts (1.8×10² PFU's/ml) in the nose (FIGS. 18A and 19). Significant differences in nasal shedding of BVDV virus titer were seen between different groups. On 6 dpc and 8 dpc, all five control animals shed virus, and one animal (#638), shed relatively high amounts of the virus on 6 dpc and 8 dpc, 5.5×10² PFU's/ml and 1.25×10² PFU's, respectively (FIG. 19). Grubb's test (generalized extreme studentized deviate method) revealed the existence of outlier in BVD-QMV2* group (Animal #630). On 11 dpc, only one control animal (#637) shed a low amount of virus (5 PFU's/ml). In the case of QMV-BVD2*, only one (#630) of four animals shed a few amounts of challenge virus on 4 dpc (5 PFU's/ml) and 6 dpc (35 PFU's/ml) (FIGS. 18A and FIG. 19). Notably, calf #630 also had the lowest leukocyte count among all the groups on the day of the challenge (6.9×110³), which is at the lower end of a normal range (6-12×10³) and 2×10³ fewer than the mean (FIG. 17).

None of the “Bovi” vaccinees shed the challenge virus at a detectable level by plaque assay. With the exception of one calf (#630), the QMV-BVD2* vaccinees did not shed the challenge virus. Nevertheless, as a whole, compared with the sham-vaccinated control calves, nasal virus shedding in the QMV-BVD2*-vaccinated calves were reduced significantly (FIGS. 18A and 19).

### 3.11 Both QMV-BVD2* and “Bovi” Vaccinated Groups Did Not Have Viremia Upon Challenge (With the Exception of One Outlier in the QMV-BVD2* Group

FIG. 18 shows BVDV nasal virus shedding and viremia following challenge with BVDV2 890 strain. In A, virus isolated from the nasal swabs of each animal following the challenge with BVDV 2 non-cytopathic strain 890 was titrated in MDBK cells by plaque assay as described in the materials methods for the cytopathic strain 125. However, the viral plaques were visualized after 96 h post-inoculation using BVD2-E2-specific Mab and fluorescent-tagged secondary antibody. Shown are the titers of each animal of the three vaccination groups on 0 dpc, 4 dpc, 6 dpc, 8 dpc and 11 dpc. The data represent mean+individual values in each group. Significant differences in nasal shedding of BVDV virus titer were seen between different groups. n=5 (QMV-BVD2* group contained 4 animals); Two-way ANOVA followed by Bonferroni post-tests to compare replicate means by row; *p<0.05, **p<0.01, *** p<0.001. Grubb's test (generalized extreme studentized deviate method) revealed the existence of outlier in BVD-QMV2* group (Animal #630). On both 4 and 8 dpc, Calf #630 in BVD-QMV2* group was a significant outlier (p<0.05) for nasal virus shedding, in comparison to other calves in the same group (FIG. 19). In B, BVDV viremia was assessed by Real-time quantitative RT-PCR using the VetMax-Gold BVDV kit (ThermoFisher, #4413938) after the challenge with 890. BVDV2 genomic copy numbers were calculated according to CT-values of a standard curve. Shown is the mean copy number of the BVDV genome in one ng total RNA of three independent PCR analyses in duplicates of each animal of the three vaccination groups on 0 dpc, 4 dpc, 6dpc, 8 dpc and 11 dpc. The dotted line separates negative from positive results and is based on the highest copy number detected in samples from 0 dpc (Threshold of 2.32 copies/ng total RNA). The data represent mean +individual values in each group. Significant differences in BVDV genome copies were seen between different groups. n=5 (QMV-BVD2* group contained 4 animals); Two-way ANOVA followed by Bonferroni post-tests to compare replicate means by row; **p<0.01. As above in “A”, according to Grubb's test—generalized extreme studentized deviate method, on 4, 6 and 8 dpc, Calf #630 in BVD-QMV2* group was a significant outlier for viremia (p<0.05) in comparison to other calves in the group (FIG. 20).

BVDV viremia was assessed by detecting BVDV genomic copies in PBMCs by RT-qPCR. As depicted in FIG. 18B, the dotted line separates negative from positive results and is based on the highest copy number detected in samples from 0 dpc (Threshold of 2.32 copies/ng total RNA). All control animals had viremia for 8 days, starting on 4 dpc until 11 dpc (the last day of assessment). The average copy number of the BVDV genome/ng total RNA determined for the unvaccinated control animals was 1.36 (0 dpc), 13.3 on 4 dpc, 272.26 on 6 dpc, 677.6 on 8pc, and 47.8 on 11 dpc (the last test performed) (FIGS. 18B and 20). Significant differences in BVDV genome copies were seen between different groups n=5 (QMV-BVD2* group contained 4 animals). Two-way ANOV followed by Bonferroni post-tests were performed to compare means by row; *p<0.05, **p<0.01 and ***p<0.001.

In the case of QMV-BVD2* vaccinated calves, the corresponding mean genome copy numbers were1.02 (0 dpc), 5.88 (4 dpc), 57.97 (6 dpc), 6.52 (8 dpc), and 1. 73 (11 dpc) (FIGS. 18B and 20). For the “Bovi” treatment group, the corresponding genome copy numbers were 1.16 (0 dpc), 1.71 (4 dpc), 2.29 (6 dpc), 2.28 (8 dpc) and 1.78 (11 dpc) (FIGS. 18B and 20). However, when the genomic copy number in the individual calves belonging to the “Bovi” and QMV-BVD2* was analyzed, it was revealed that with the exception the calf #630, which had 13.93, 57.97, and 20.04 BVDV genomic copies on 4, 6, and 8 dpc, respectively, the rest of the three calves in the group had much lower genomic copy numbers. Calf #630 in BVD-QMV2* group was a significant outlier for viremia (p<0.05) in comparison to other calves in the group (FIG. 20)). Notably, during 4dpc-11 dpc, the genomic copy numbers in the PBMC's of the other three calves in the QMV-BVD2* group were comparable to those in the “Bovi” treatment group (FIG. 20).

As noted above, animal #630 (QMV-BVD2* group) also had a low leukocyte count (6.9×10³/μl) on the day of the challenge (FIG. 17). Therefore, compared with the sham-vaccinated control calves, the reduction in viremia in both the QMV-BVD2*- and “Bovi”-vaccinated calves were significant (FIGS. 18B and 20).

### 3.12. Both QMV-BVD2* and “Bovi” Vaccinated Calves were Clinically Protected After BVDV-2 Challenge

After the challenge with BVDV2 until the day of euthanasia (20 dpc), clinical signs were recorded daily, based on the criteria listed in FIG. 23. Rectal temperature (FIG. 22), nasal and ocular discharge, lethargy, dyspnea, cough, mucosal or oral lesions, anorexia, and diarrhea were recorded to determine the clinical score for each calf (FIG. 24). Rectal temperature was measured with a digital thermometer on the indicated days (FIG. 22). FIG. 21A shows the mean temperature of each treatment group with standard deviation. Significant differences in rectal temperature were seen between different groups. n=5 (QMV-BVD2* group contained 4 animals); Two-way ANOVA followed by Bonferroni post-tests to compare replicate means by row; *p<0.05, ***p<0.001 between Control and Bovi group. ^{#}p<0.05 between Bovi and QMV-BVD2* group. ^{$}p<0.05, ^{$$$}p<0.001 between Control and QMV-BVD2* group. All animals from the unvaccinated control group developed a moderate (≥40° C., ¼ animals) to severe fever (≥40.5° C., ⅘ animals) on or after 6 dpc, which lasted for several days (FIGS. 21A and 22). Two calves in the “Bovi” group (#627 and #628) had mild (39.5-39.9° C.) to moderate (40.0-40.4° C.) non-specific fever during 3-6 dpc (FIG. 22). Two calves (#629 and #632) had a mild non-specific fever during 0-5 dpc, and one calf (#635) had a normal (38.5° C.-39.2° C.) rectal temperature (FIG. 22). In the QMV-BVD2* group, one calf (#630), designated above as an outlier for nasal virus shedding and viremia (sections 3.10 and 3.11) had a moderate fever for three days (5-8 dpc), and another calf (#640) had a moderate fever (40.0° C.) on 6 dpc but was normal the next day (FIG. 22). The remaining two calves (#633 and #639) remained normal until the day of euthanasia.

Following the challenge, all control animals (5/5) also showed other clinical signs, in addition to fever, associated with the BVDV infection, i.e., nasal discharge, mild coughing, lethargy, anorexia, and diarrhea, which were scored according to the criteria listed in FIG. 23. The mean clinical scores obtained for each of the three treatment groups following the challenge until 14 dpc are shown in FIG. 21 B. Significant differences in clinical signs score were seen between different groups. n=5 (QMV-BVD2* group contained 4 animals); Two-way ANOVA followed by Bonferroni post-tests to compare replicate means by row; *p<0.05, ** p<0.01 ***p<0.001 between Control and Bovi group. ^{$$}p<0.01, ^{$$$}p<0.001 between Control and QMV-BVD2* group. On 8 dpc, two control calves had mild to moderate nasal discharge, and the following day (9 dpc), all animals in the control group (5/5) had moderate to severe lethargy and anorexia (FIG. 24). Two of the control animals also had diarrhea from 9 dpc until 13 dpc. In contrast, none of the calves vaccinated with QMV-BVD2* or “Bovi” commercial vaccine showed any of these signs noted above (FIGS. 21B and 24).

### 3.13. Based on Gross and Histopathological Lesions in the Lungs, the QMV-BVD2* Vaccine Protected the Calves Better than the “Bovi” Vaccine

### 3.13.1. Gross Lesions

No gross lesions were found in the lungs of the QMV- BVD2* vaccines (data not shown). Three of the five calves in the control (sham-vaccinated) group had gross lesions. The control group's lesions consisted of diffuse reddening and consolidation of the right cranial and cranial portion of the left cranial lung lobes in 2 calves (FIG. 25). One control (#645), a cranioventral consolidation of the lungs could be observed and were outlines by arrows on FIG. 25 (left panel). This portion is consolidated with a classical pattern of acute bronchopneumonia. In the third calf, the cranioventral portions of the lungs were reddened, heavy and wet, but not consolidated (not shown). Two of the 5 “Bovi” vaccinees had detectable gross lesions. One (#635) had bilateral reddening of the cranial lobes and the right middle lobe. There was a consolidation of individual lobules along the right middle and right cranial lung lobes along the ventral margins, with sparing of adjacent lobules as shown with arrows on the middle panel of FIG. 25. No consolidation was detected in the left lung. The second calf (#628) had reddening of the right middle and bilateral cranial lobes in an irregular lobular pattern as delineated with the circle. Histologically, the latter area had mild, multifocal acute inflammation. Arrows delineate extensively consolidated and fibrotic right cranial lobe. Affected areas were heavy and wet but minimally consolidated (FIG. 25, right panel).

### 3.13.2. Histopathology Findings

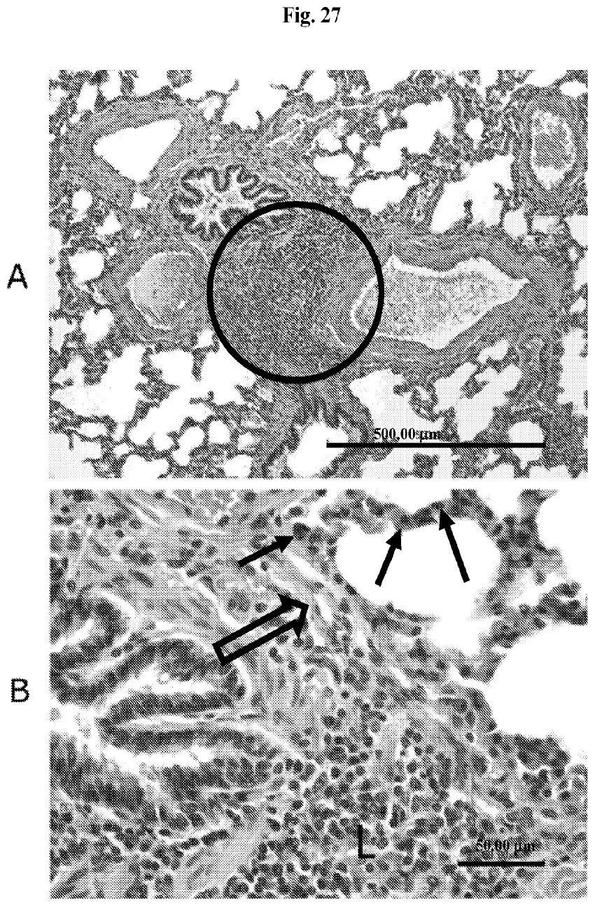

FIGS. 26-28. show histopathology of representative sections of lung tissues showing lesions from control (FIGS. 26A and 26B), “Bovi” (FIGS. 27A and 27B), and QMV-BVD2* (FIGS. 28A and 28B) vaccinated groups. FIG. 26A shows that the lung of a control group calf has substantial bronchiolar damage. The open arrow shows fibrosis filling the lumen of the affected bronchiole (bronchiolitis obliterans). A lymphoplasmacytic cuff surrounding the bronchioles (single solid arrow) and smooth muscle hypertrophy and fibrosis (double arrows). Bar=500 μm. FIG. 26B. shows that the bronchiole of a control group calf is surrounded by a cuff of mononuclear cells (L), plasma cells, lymphocytes, and fewer macrophages. Neutrophilic transmucosal exocytosis with accumulation in the bronchiolar lumen (double arrows) indicates that the inflammation remains active. (Bar=50 μm). FIG. 27A shows the histopathology of lung tissues of a “Bovi”-vaccinated calf. The marked increase in Low-grade B-cell bronchial associated lymphoid tissue (BALT) is noticeable with bronchus-associated lymphoid tissues as delineated by a circled. The bronchiole is constricted, which is not considered a significant lesion. Alveolar spaces are clear. Bar=500 μm. FIG. 27B shows a marked increase in BALT (L). Mild fibrosis is evident as shown with open arrow. A few mononuclear inflammatory cells have infiltrated the adjacent alveolar septa as shown with solid arrows). Bar=50 μm. Noticeably, FIG. 28A shows minimal bronchiolar change with few peribronchiolar lymphocytes and plasma cells as emphasized arrows in a QMV-BVD2* vaccinated calf. Alveolar spaces are clear, and septa are normal. Bar=500 μm. FIG. 28B shows that plasma cells (arrow) and lymphocytes are present in the peribronchiolar interstitium in approximately normal numbers in a QMV-BVD2*-vaccinated calf. The smooth muscle may be mildly hypertrophied, but it also may be an artifact of perimortem contraction. Bar=50 μm.

No significant differences were found among treatment groups in the histological scores of any tissues other than the lungs. The most consistent lesion in the lungs was an increase in peribronchial lymphocytes, either follicular or diffuse, with thick peribronchial cuffs in the most severely affected sections, especially in control and “Bovi” treatment groups. All groups had some degree of peribronchial fibrosis. All had inconsistent transmucosal neutrophilic exocytosis and some excess mucus within bronchi and/or bronchioles (data not shown). However, only the “Bovi” and control groups had intraluminal neutrophils and rarely had bronchiolitis obliterans (2 of 5 in control and 1 of 5 in “Bovi”) (FIGS. 26A, 26B, 27A and 27B). The most common parenchymal lesion seen in all groups was complete atelectasis of a portion or entire lobules, most likely caused by euthanasia. There were alveolar infiltrates in the “Bovi” and control groups, which were either histiocytic or mixed, neutrophilic, and histiocytic (FIGS. 26A, 26B, 27A and 27B). The infiltrates were limited and multifocal in the “Bovi” group (FIGS. 27A and 27B) but more widespread in some sections from the control group (FIGS. 26A and 26B). No alveolar infiltrates were present in the QMV-BVD2* group (FIGS. 28A and 28B) Septal and/or pleural lesions of variable edema or fibrosis and mild lymphocytic infiltration were present in all groups.

Histological lesions seen in the other tissues were expected. Lymphoid hyperplasia with occasional early depletion was seen in most lymphoid organs (data not shown). Sinus histiocytosis was often present. Minimal to mild lesions of chronic interstitial inflammation in the kidneys and minimal portal inflammation in the livers were common in all groups (data not shown).

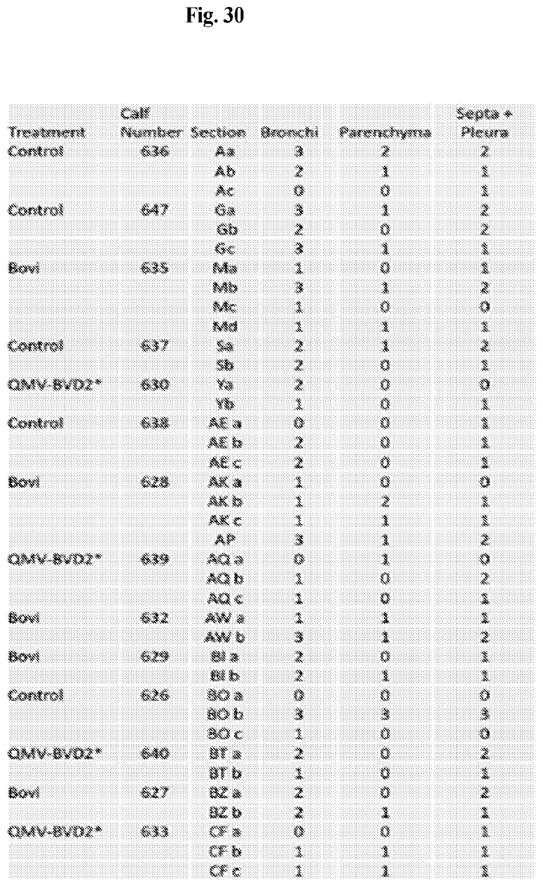

FIG. 30 provides lung lesion score for individual animal and FIG. 29 provides a graph representation of the mean histopath-lesion scores across treatment groups by areas of lung tissues. In FIG. 29, for each tissue, the left column shows the mean score of lesions in the “Bovi”-vaccinated group, the middle column shows the mean score of lesions in the control group and the right column shows the mean score in the QMV BVD2*-vaccinated group. The results of the statistical analysis indicated that there were significant differences regarding vaccine efficacy across the three groups (p<0.049, ηₚ²=0.42). Specifically, a significant difference was detected between the QMV-BVD2*-vaccinated and control groups in Bronchi (p<0.05). In other words, QMV-BVD2* (M=1, SD=0.67) protected the calves significantly better for lung lesions after the BVDV-2 challenge when compared with the calves in the control group (M=1.76, SD=1.12) (FIG. 29).

### References

1. USDA, United States Department of Agriculture. Part I: Reference of dairy health and management practices in the United States. In APHIS: VS, CEAH, National Animal Health Monitoring System: . Fort Collins (CO): USDA, 2007.
2. Fulton, R. W.; Cook, B. J.; Step, D. L.; Confer, A. W.; Saliki, J. T.; Payton, M. E.; Burge, L. J.; Welsh, R. D.; Blood, K. S., Evaluation of health status of calves and the impact on feedlot performance: assessment of a retained ownership program for postweaning calves. Can J Vet Res 2002, 66, (3), 173-80.
3. Taylor, J. D.; Fulton, R. W.; Lehenbauer, T. W.; Step, D. L.; Confer, A. W., The epidemiology of bovine respiratory disease: what is the evidence for preventive measures? Can Vet J2010, 51, (12), 1351-9.
4. Koppers-Lalic, D.; Reits, E. A.; Ressing, M. E.; Lipinska, A. D.; Abele, R.; Koch, J.; Marcondes Rezende, M.; Admiraal, P.; van Leeuwen, D.; Bienkowska-Szewczyk, K.; Mettenleiter, T. C.; Rijsewijk, F. A.; Tampe, R.; Neefjes, J.; Wiertz, E. J., Varicelloviruses avoid T cell recognition by UL49.5-mediated inactivation of the transporter associated with antigen processing. Proc Natl Acad Sci USA 2005, 102, (14), 5144-9.
5. Koppers-Lalic, D.; Verweij, M. C.; Lipinska, A. D.; Wang, Y.; Quinten, E.; Reits, E. A.; Koch, J.; Loch, S.; Marcondes Rezende, M.; Daus, F.; Bienkowska-Szewczyk, K.; Osterrieder, N.; Mettenleiter, T. C.; Heemskerk, M. H.; Tampe, R.; Neefjes, J. J.; Chowdhury, S. I.; Ressing, M. E.; Rijsewijk, F. A.; Wiertz, E. J., Varicellovirus UL 49.5 proteins differentially affect the function of the transporter associated with antigen processing, TAP. PLoS Pathog 2008, 4, (5), e1000080.
6. Winkler, M. T.; Doster, A.; Jones, C., Bovine herpesvirus 1 can infect CD4(+) T lymphocytes and induce programmed cell death during acute infection of cattle. J Virol 1999, 73, (10), 8657-68.
7. Bryant, N. A.; Davis-Poynter, N.; Vanderplasschen, A.; Alcami, A., Glycoprotein G isoforms from some alphaherpesviruses function as broad-spectrum chemokine binding proteins. EMBO J 2003, 22, (4), 833-46.
8. Chase, C. C.; Thakur, N.; Darweesh, M. F.; Morarie-Kane, S. E.; Rajput, M. K., Immune response to bovine viral diarrhea virus--looking at newly defined targets. Anim Health Res Rev 2015, 16, (1), 4-14.
9. Jones, C.; Chowdhury, S., A review of the biology of bovine herpesvirus type 1 (BoHV-1), its role as a cofactor in the bovine respiratory disease complex and development of improved vaccines. Anim Health Res Rev 2007, 8, (2), 187-205.
10. Ridpath, J., The contribution of infections with bovine viral diarrhea viruses to bovine respiratory disease. Vet Clin North Am Food Anim Pract 2010, 26, (2), 335-48.
11. Jones, C.; Chowdhury, S., Bovine herpesvirus type 1 (BHV-1) is an important cofactor in the bovine respiratory disease complex. Vet Clin North Am Food Anim Pract 2010, 26, (2), 303-21.
12. Iotti, B.; Valdano, E.; Savini, L.; Candeloro, L.; Giovannini, A.; Rosati, S.; Colizza, V.; Giacobini, M., Farm productive contexts and the dynamics of bovine viral diarrhea (BVD) transmission. Prev Vet Med 2019, 165, 23-33.
13. Moennig, V.; Becher, P., Control of Bovine Viral Diarrhea. Pathogens 2018, 7, (1).
14. Wei, H.; Wang, Y.; Chowdhury, S. I., Bovine herpesvirus type 1 (BHV-1) UL49.5 luminal domain residues 30 to 32 are critical for MHC-I down-regulation in virus-infected cells. PLoS One 2011, 6, (10), e25742.
15. O'Rourke, K., BVDV: 40 years of effort and the disease still has a firm hold. J Am Vet Med Assoc 2002, 220, (12), 1770-3.
16. Peterhans, E.; Jungi, T. W.; Schweizer, M., [How the bovine viral diarrhea virus outwits the immune system]. Dtsch Tierarztl Wochenschr 2006, 113, (4), 124-9.
17. Roth, J. A.; Kaeberle, M. L., Suppression of neutrophil and lymphocyte function induced by a vaccinal strain of bovine viral diarrhea virus with and without the administration of ACTH. Am J Vet Res 1983, 44, (12), 2366-72.
18. Rajput, M. K. S.; Abdelsalam, K.; Darweesh, M. F.; Braun, L. J.; Kerkvliet, J.; Hoppe, A. D.; Chase, C. C. L., Both cytopathic and non-cytopathic bovine viral diarrhea virus (BVDV) induced autophagy at a similar rate. Vet Immunol Immunopathol 2017, 193-194, 1-9.
19. van Oirschot, J. T.; Bruschke, C. J.; van Rijn, P. A., Vaccination of cattle against bovine viral diarrhoea. Vet Microbiol 1999, 64, (2-3), 169-83.
20. Ridpath, J. F., Bovine viral diarrhea virus: global status. Vet Clin North Am Food Anim Pract 2010, 26, (1), 105-21, table of contents.
21. O'Toole, D.; Van Campen, H., Abortifacient vaccines and bovine herpesvirus-1. J Am Vet Med Assoc 237, (3), 259-60.
22. Jones, C.; Chowdhury, S., Bovine herpesvirus type 1 (BHV-1) is an important cofactor in the bovine respiratory disease complex. Vet Clin North Am Food Anim Pract 26, (2), 303-21.
23. Martinod, S., Risk assessment related to veterinary biologicals: side-effects in target animals. Rev Sci Tech 1995, 14, (4), 979-89.
24. Lorenz, I.; Earley, B.; Gilmore, J.; Hogan, I.; Kennedy, E.; More, S. J., Calf health from birth to weaning. III. housing and management of calf pneumonia. Ir Vet J 64, (1), 14.
25. O'Toole, D.; Miller, M. M.; Cavender, J. L.; Cornish, T. E., Pathology in practice: abortion in the heifers of this report was a result of BoHV-1 infection. J Am Vet Med Assoc 241, (2), 189-91.
26. Kasonta, R.; Sauter-Louis, C.; Holsteg, M.; Duchow, K.; Cussler, K.; Bastian, M., Effect of the vaccination scheme on PregSure(R) BVD induced alloreactivity and the incidence of Bovine Neonatal Pancytopenia. Vaccine 2012, 30, (47), 6649-55.
27. Deutskens, F.; Lamp, B.; Riedel, C. M.; Wentz, E.; Lochnit, G.; Doll, K.; Thiel, H. J.; Rumenapf, T., Vaccine-induced antibodies linked to bovine neonatal pancytopenia (BNP) recognize cattle major histocompatibility complex class I (MHC I). Vet Res 2011, 42, 97.
28. Kaashoek, M. J.; Rijsewijk, F. A.; Ruuls, R. C.; Keil, G. M.; Thiry, E.; Pastoret, P. P.; Van Oirschot, J. T., Virulence, immunogenicity and reactivation of bovine herpesvirus 1 mutants with a deletion in the gC, gG, gI, gE, or in both the gI and gE gene. Vaccine 1998, 16, (8), 802-9.
29. EFSA, Scientific committee on animal health and animal wellfare report on bovine herpesvirus (BHV-1) marker vaccines and the and the accompanying diagnostic tests. 2000, (Adopted Oct. 25, 2000).
30. Chowdhury, S. I.; Wei, H.; Weiss, M.; Pannhorst, K.; Paulsen, D. B., A triple gene mutant of BoHV-1 administered intranasally is significantly more efficacious than a BoHV-1 glycoprotein E-deleted virus against a virulent BoHV-1 challenge. Vaccine 2014, 32, (39), 4909-15.
31. Chowdhury, S. I., Identification of an epitope within the Bovine herpesvirus 1 glycoprotein E cytoplasmic tail and use of a monoclonal antibody directed against the epitope for the differentiation between vaccinated and infected animals. J Virol Methods 2016, 233, 97-104.
32. Shi, Y.; Liu, C. H.; Roberts, A. I.; Das, J.; Xu, G.; Ren, G.; Zhang, Y.; Zhang, L.; Yuan, Z. R.; Tan, H. S.; Das, G.; Devadas, S., Granulocyte-macrophage colony-stimulating factor (GM-CSF) and T-cell responses: what we do and don't know. Cell Res 2006, 16, (2), 126-33.
33. Lokhandwala, S.; Fang, X.; Waghela, S. D.; Bray, J.; Njongmeta, L. M.; Herring, A.; Abdelsalam, K. W.; Chase, C.; Mwangi, W., Priming Cross-Protective Bovine Viral Diarrhea Virus-Specific Immunity Using Live-Vectored Mosaic Antigens. PLoS ONE 2017, 12, (1), e0170425.
34. Chowdhury, S. I.; Mahmood, S.; Simon, J.; Al-Mubarak, A.; Zhou, Y., The Us9 gene of bovine herpesvirus 1 (BHV-1) effectively complements a Us9-null strain of BHV-5 for anterograde transport, neurovirulence, and neuroinvasiveness in a rabbit model. J Virol 2006, 80, (9), 4396-405.
35. Al-Mubarak, A.; Simon, J.; Coats, C.; Okemba, J. D.; Burton, M. D.; Chowdhury, S. I.,

Glycoprotein E (gE) specified by bovine herpesvirus type 5 (BHV-5) enables trans-neuronal virus spread and neurovirulence without being a structural component of enveloped virions. Virology 2007, 365, (2), 398-409.
36. Chowdhury, S. I.; Lee, B. J.; Ozkul, A.; Weiss, M. L., Bovine herpesvirus 5 glycoprotein E is important for neuroinvasiveness and neurovirulence in the olfactory pathway of the rabbit. J Virol 2000, 74, (5), 2094-106.
37. Hutchings, D. L.; van Drunen Littel-van den Hurk, S.; Babiuk, L. A., Lymphocyte proliferative responses to separated bovine herpesvirus 1 proteins in immune cattle. J Virol 1990, 64, (10), 5114-22.
38. Ridpath, J. F.; Neill, J. D.; Peterhans, E., Impact of variation in acute virulence of BVDV1 strains on design of better vaccine efficacy challenge models. Vaccine 2007, 25, (47), 8058-66.
39. Njongmeta, L. M.; Bray, J.; Davies, C. J.; Davis, W. C.; Howard, C. J.; Hope, J. C.; Palmer, G. H.; Brown, W. C.; Mwangi, W., CD205 antigen targeting combined with dendritic cell recruitment factors and antigen-linked CD40L activation primes and expands significant antigen-specific antibody and CD4(+) T cell responses following DNA vaccination of outbred animals. Vaccine 2012, 30, (9), 1624-35.
40. Woobbrock, J. O.; Findlater, L.; Gergle, D.; Huggins, J. J., The algned rank transform for nonparametric factorial analyses using only anova procedures. In Proceedings of the SIGCHI conference on human factors in computing systems. 2011, pp.143-146.
41. Feys, J., New nonparametric rank tests for interactions in factorial designs with repeated measures. Journal of Modern Applied Statistical Methods 2016, 15 (1), 78-99.
42. Purtle, L.; Mattick, D.; Schneider, C.; Smith, L.; Xue, W.; Trigo, E., One year duration of immunity of the modified live bovine viral diarrhea virus type 1 and type 2 and bovine herpesvirus-1 fractions of Vista(R) Once SQ vaccine. Vaccine 2016, 34, (13), 1582-1588.
43. Stahl, K.; Alenius, S., BVDV control and eradication in Europe—an update. Jpn J Vet Res 2012, 60 Sunni, S31-9.
44. Lindberg, A. L.; Alenius, S., Principles for eradication of bovine viral diarrhoea virus (BVDV) infections in cattle populations. Vet Microbiol 1999, 64, (2-3), 197-222.
45. Presi, P.; Heim, D., BVD eradication in Switzerland—a new approach. Vet Microbiol 2010, 142, (1-2), 137-42.
46. Bachofen, C.; Stalder, H.; Vogt, H. R.; Wegmuller, M.; Schweizer, M.; Zanoni, R.; Peterhans, E., [Bovine viral diarrhea (BVD): from biology to control]. Berl Munch Tierarztl Wochenschr 2013, 126, (11-12), 452-61.
47. Wernike, K.; Schirrmeier, H.; Strebelow, H. G.; Beer, M., Eradication of bovine viral diarrhea virus in Germany-Diversity of subtypes and detection of live-vaccine viruses. Vet Microbiol 2017, 208, 25-29.
48. Schmitt, J.; Becher, P.; Thiel, H. J.; Keil, G. M., Expression of bovine viral diarrhoea virus glycoprotein E2 by bovine herpesvirus-1 from a synthetic ORF and incorporation of E2 into recombinant virions. J Gen Virol 1999, 80 (Pt 11), 2839-2848.
49. Kweon, C. H.; Kang, S. W.; Choi, E. J.; Kang, Y. B., Bovine herpes virus expressing envelope protein (E2) of bovine viral diarrhea virus as a vaccine candidate. J Vet Med Sci 1999, 61, (4), 395-401.

1. A bovine herpesvirus-1 (BoHV-1) recombinant vector comprising a deletion of a cytoplasmic tail of envelope glycoprotein gE (gE-CT), a truncation of glycoprotein gG, a deletion of envelope protein UL49.5 amino acid residues 30-32, and a deletion of UL49.5 cytoplasmic tail amino acid residues 80-96.

2. (canceled)

3. The BoHV-1 recombinant vector of claim 1, wherein the truncation of glycoprotein gG comprises a deletion of amino-terminal amino acid residues 1-67.

4. (canceled)

5. The BoHV-1 recombinant vector of claim 2, wherein the truncated sequence of the glycoprotein gG is replaced by a sequence having at least 90% sequence identity with the sequence SEQ ID NO:3.

6. The BoHV-1 recombinant vector of claim 1, further comprising a sequence having at least 90% sequence identity with a sequence selected from SEQ ID NO: 7 in combination with SEQ ID NO: 8 or SEQ ID NO: 10 or SEQ ID NO: 7 in combination with SEQ ID NO: 10.

7. The BoHV-1 recombinant vector of claim 1, further comprising at least two heterologous antigens derived from viral envelope glycoproteins inserted therein.

8. The BoHV-1 recombinant vector of claim 7, wherein the at least two heterologous antigens are from the same or different viruses selected from Bovine Viral Diarrhea Virus type 1 (BVDV-1), Bovine Viral Diarrhea Virus type 2 (BVDV-2), Bovine Herpesvirus-1 (BoHV-1), Bovine Respiratory Syncytial Virus (BRSV), Rift Valley Fever Virus (RVFV).

9. The BoHV-1 recombinant vector of claim 7, wherein the at least two heterologous antigens are selected from BVDV-2 E2, BVDV-2 Erns, BRSV F, BRSV G, RVFV Gn, RFVF Gc, a polypeptide having at least 90% sequence identity with the polypeptide sequences defined as SEQ ID NO: 11, and a polypeptide having at least 90% sequence identity with the polypeptide sequences defined as SEQ ID NO: 12.

10-12. (canceled)

13. The BoHV-1 recombinant vector of claim 7, wherein at least one of the at least two heterologous antigens is expressed as a fusion protein with a fusion partner.

14-15. (canceled)

16. The BoHV-1 recombinant vector of claim 13, wherein the fusion partner is selected from a cytokine, a gD signal sequence, a V5 epitope, a histidine tail, GM-CSF, or any combination thereof.

17. The BoHV-1 recombinant vector of claim 7, wherein at least one of the at least two heterologous antigens is expressed from a heterologous promoter.

18. (canceled)

19. The BoHV-1 recombinant vector of claim 17 wherein at least one of the at least two heterologous antigens is expressed from a HCMV promotor, a human elongation factor 1 alpha promotor, a CMV IE promotor, or a CAG synthetic promotor.

20. A composition comprising a carrier and at least one BoHV-1 recombinant vector according to claim 1.

21. (canceled)

22. A method for treating a mammal having or at risk of having a viral infection, in particular a viral respiratory infection, by administering at least one BoHV-1 recombinant vector of claim 1 to the mammal.

23. (canceled)

24. The method of claim 22, wherein the viral infection is caused by at least one of the viruses selected from BVDV-1, BVDV-2, BoHV-1, BRSV and RVFV.

25-29. (canceled)

30. A live attenuated vaccine for protection against at least one Bovine viral disease, in particular a Bovine viral respiratory infection, comprising at least one of the BoHV-1 recombinant vector according to claim 1.

31. The vaccine of claim 30, wherein the Bovine viral respiratory infection is caused by at least one of the viruses selected from BVDV-1, BVDV-2, BoHV-1, BRSV and RVFV.

32. The vaccine of claim 31, wherein the at least one BoHV-1 recombinant vector comprises a sequence having at least 90% sequence identity with a sequence selected from SEQ ID NO: 7 in combination with SEQ ID NO: 8 or SEQ ID NO: 10 or SEQ ID NO: 7 in combination with SEQ ID NO: 10.

33. The vaccine of claim 32, wherein the RVFV antigens comprise a polypeptide having at least 90% sequence identity with the polypeptide sequences defined as SEQ ID NO: 11 and a polypeptide having at least 90% sequence identity with the polypeptide sequences defined as SEQ ID NO: 12.

34. A vaccine composition, comprising the vaccine of claim 30 and a pharmaceutically acceptable vehicle or adjuvant.

35. A method of vaccinating a cow against a BVDV infection, said method comprising inoculating the cow with the vaccine of claim 30.

36. (canceled)
